(19) United States
(12) Patent Application Publication
(10) Pub. No.: US 2025/0101437 A1
(43) Pub. Date: Mar. 27, 2025

(54) APTAMERS FOR DETECTING VARIANTS OF A TARGET ANALYTE, METHODS OF MAKING AND USES THEREOF

(71) Applicant: McMaster University, Hamilton (CA)

(72) Inventors: Yingfu Li, Dundas (CA); John Brennan, Dundas (CA); Zijie Zhang, Hamilton (CA); Jiuxing Li, Hamilton (CA); Jimmy Gu, Mississauga (CA); Dawn White, La Salle (CA); Bruno Salena, Ancaster (CA); Matthew Miller, Hamilton (CA); Deborah Yamamura, Burlington (CA)

(21) Appl. No.: 18/728,342

(22) PCT Filed: Jan. 11, 2023
(86) PCT No.: PCT/CA2023/050029

§ 371 Date: Jul. 11, 2024

Related U.S. Application Data
(60) Provisional application No. 63/298,381, filed on Jan. 11, 2022.

(51) Int. Cl. C12N 15/115; C12N 15/10; C40B 30/04; G01N 33/569

(52) U.S. Cl. CPC C12N 15/115; C12N 15/1048; C40B 30/04; G01N 33/56983; C12N 2310/16; C12N 2320/13; G01N 2333/165; G01N 2469/10

(57) ABSTRACT

This disclosure relates to a method of identifying or producing an aptamer capable of binding to at least two target analyte variants, the method comprises generating a mixture of systematic evolution of ligands by exponential enrichment (SELEX)-selected aptamers by at least one-round of SELEX with a SELEX-compatible aptamer pool against the at least two target analyte variants in parallel or sequentially, sequencing the mixture of SELEX-selected aptamers that form complexes with each of the at least two target analyte variants by high-throughput sequencing, aligning the mixture of SELEX-selected aptamers that form complexes with each of the at least two target analyte variants by sequence alignment analysis, and identifying or producing the aptamer capable of binding to the at least two target analyte variants. Aptamers thereof and uses of the aptamers thereof are also disclosed.

### RELATED APPLICATION

This disclosure claims benefit and priority of U.S. Provisional Patent Application Ser. No. 63/298,381 filed Jan. 11, 2022, which is incorporated herein by reference in its entirety.

### INCORPORATION OF SEQUENCE LISTING

A computer readable form of the Sequence Listing “3244-P67236PC00_SequenceListing.xml” (124,468 bytes) was created on Jan. 9, 2023, is filed herewith by electronic submission and is incorporated by reference herein.

### FIELD

The present disclosure relates to the field of nucleic acid aptamers, and in particular, to aptamers capable of binding variants of a target analyte, methods of making and uses thereof.

### BACKGROUND

The COVID-19 pandemic, caused by severe acute respiratory syndrome coronavirus 2 (SARS-CoV-2), has become a huge burden to our society.[1] The initial virus was discovered in December, 2019 in the city of Wuhan, China, then spread globally. During the past 18 months, several variants of concern (VoCs) have emerged, the most notable VoCs being B.1.1.7 (the UK variant; Alpha), B.1.351 (the South Africa variant; Beta), P.1 (the Brazil variant; Gamma), B.1.429 (the California variant; Epsilon) and B.1.617.2 (the Indian variant; Delta) [2] and very recently B.1.1.529 (Omicron).[3]

Large-scale testing, contact tracing and isolation have been implemented as one method to control the spread of SARS-CoV-2. However, current testing using quantitative reverse-transcription real-time polymerase chain reaction (qRT-PCR), while highly sensitive and specific for detecting SARS-CoV-2, suffers from a slow turnaround time and high cost, making it unsuitable as a screening tool to enable rapid responses to outbreaks.[4] Several commercial Antigen (Ag) tests, such as the Abbott PanBio™ COVID-19 Ag test, the Abbott BINAX Nowυ COVID-19 Ag test, and the Ellume rapid COVID-19 test, are relatively rapid and inexpensive, but are inherently less sensitive than RT-PCR assays.[5,6] For example, the sensitivity of the Abbott PanBio™ COVID-19 Ag test was found to be only 57.7% and 2.6%, respectively, using throat and saliva swabs.[7] In addition, known rapid tests have been reported to be insufficiently sensitive to detect the Omicron variant until several days after becoming infectious, showing that rapid tests are deficient in detecting emerging variants. With constant mutations of the genome of SARS-CoV-2 and rapid emergence of new VoCs, there is an urgent need for simpler, faster, more cost-effective large-scale testing methods that work with both current and emerging VoCs.

The background herein is included solely to explain the context of the disclosure. This is not to be taken as an admission that any of the material referred to was published, known, or part of the common general knowledge as of the priority date.

### SUMMARY

DNA aptamers, which can be selected from random sequence pools by in vitro selection (SELEX),[8,9] offer several key advantages over antibodies for the development of rapid tests, such as small size, high chemical and thermal stability, easy and precise modification, scalable production and minimal batch-to-batch variation.[10] In addition, they have been shown to be capable of detecting targets present in clinical samples.[11-13] These attractive properties make aptamers important molecular recognition elements (MREs) for assay and diagnostic development.[14-16]

In accordance with an aspect, there is provided a method to identify or produce aptamers that universally (or non-discriminatively) recognize target analytes variants.

Herein provided is a method of identifying or producing an aptamer capable of binding to at least two target analyte variants, the method comprising:
a) generating a mixture of systematic evolution of ligands by exponential enrichment (SELEX)-selected aptamers by at least one-round of SELEX with a SELEX-compatible aptamer pool against the at least two target analyte variants in parallel or sequentially;
b) sequencing the mixture of SELEX-selected aptamers that form complexes with each of the at least two target analyte variants by high-throughput sequencing;
c) aligning the mixture of SELEX-selected aptamers that form complexes with each of the at least two target analyte variants by sequence alignment analysis; and
d) identifying or producing the aptamer capable of binding to the at least two target analyte variants.

In some embodiments, the method further comprises, prior to a), generating an aptamer pool compatible with SELEX. In some embodiments, the SELEX-compatible aptamer pool comprises a plurality of nucleic acid molecules comprising at least one random nucleotide domain having an aptamer-like structure, the random nucleotide domain flanked by a 5′-end region and a 3′-end region. In some embodiments, the 5′-end region hybridizes to the 3′-end region to form a duplex DNA element. In some embodiments, the 5′-end region comprises a nucleotide sequence of SEQ ID NO: 102 or 103, and the 3′-end region comprises a nucleotide sequence of SEQ ID NO: 132 or the reverse complement of SEQ ID NO: 104 or 105. In some embodiments, the SELEX-compatible aptamer pool comprises a plurality of nucleic acid molecules having the nucleotide sequence of SEQ ID NO: 101.

In some embodiments, the target analyte variant is a microorganism, a virus, and/or a molecule present in a microorganism or a virus. In some embodiments, the target analyte variant is a protein. In some embodiments, the target analyte variant is a protein from SARS-CoV-2. In some embodiments, the target analyte variant is SARS-CoV-2 spike protein. In some embodiments, the method comprises in a) generating a mixture of SELEX-selected aptamers by at least three, four, five, six, seven, eight, or nine target analyte variants.

In some embodiments, the high-throughput sequencing in b) comprises single-molecule real-time sequencing, ion semiconductor sequencing, pyrosequencing, sequencing by synthesis, combinatorial probe anchor synthesis sequencing, sequencing by ligation, nanopore sequencing, or GenapSys™ sequencing. In some embodiments, the high-throughput sequencing comprises sequencing by synthesis.

Several groups have isolated DNA aptamers that bind the S1 subunit of the spike protein of the original SARS-CoV-2 virus or its receptor-binding domain (RBD).[17-25] However, other than the finding that two S1-binding DNA aptamers, named MSA1 and MSA5, also exhibited similar affinity for the spike proteins of the B.1.1.7 variant of concern, limited information is available on whether the reported aptamers can bind the spike proteins of other important VoCs.[17]

Also provided is an aptamer comprising a 5′-end region, a 3′-end region, and a nucleotide sequence selected from the group consisting of SEQ ID NOS: 2-100, a functional fragment, and functional variant thereof, or a nucleotide sequence selected from the group consisting of SEQ ID NOS: 113-117, a functional fragment, and functional variant thereof. In some embodiments, the 5′-end region comprises a nucleotide sequence of SEQ ID NO: 102 or 103, and the 3′-end region comprises a nucleotide sequence of SEQ ID NO: 132 or the reverse complement of SEQ ID NO: 104 or 105. In some embodiments, the aptamer comprises a 5′-end region, a 3′-end region, and a nucleotide sequence selected from the group consisting of SEQ ID NOS: 2-10, a functional fragment, and functional variant thereof. In some embodiments, the aptamer comprises a nucleotide sequence comprising SEQ ID NO: 116 or 117.

Also provided is a method for detecting the presence of SARS-CoV-2 spike protein in a sample, the method comprising:
a) contacting the sample with an aptamer described herein, wherein the aptamer binds the spike protein; and
b) detecting the presence of the aptamer bound to the spike protein in the sample, wherein detection of bound aptamer indicates the presence of SARS-CoV-2 spike protein or variant thereof.

In some embodiments, the SARS-CoV-2 spike protein variant comprises B.1.1.7, B.1.351, P.1, B.1.429, B.1.617.1 B.1.617.2, B.1.617.2.1, or B.1.1.529 spike protein variant. In some embodiments, the aptamer comprises a 5′-end region, a 3′-end region, and a nucleotide sequence selected from the group consisting of SEQ ID NOS: 1-100, a functional fragment, and functional variant thereof, or comprises a nucleotide sequence selected from the group consisting of SEQ ID NOS: 106-117, a functional fragment, and functional variant thereof. In some embodiments, the aptamer comprises a 5′-end region, a 3′-end region, and a nucleotide sequence selected from the group consisting of SEQ ID NOS: 1-10, a functional fragment, and functional variant thereof. In some embodiments, the aptamer comprises a 5′-end region, a 3′-end region, and a nucleotide sequence of SEQ ID NO: 1, a functional fragment, or functional variant thereof. In some embodiments, the aptamer comprises a nucleotide sequence selected from the group consisting of SEQ ID NOS: 108, 112-117, a functional fragment, and functional variant thereof. In some embodiments, the aptamer comprises a nucleotide sequence selected from the group consisting of SEQ ID NOS: 108, 112, 116, 117, a functional fragment, and functional variant thereof. In some embodiments, the aptamer comprises a nucleotide sequence of SEQ ID NO: 112, a functional fragment, or functional variant thereof.

In some embodiments, the method detects SARS-CoV-2 infection in a subject. In some embodiments, the sample is saliva, sputum, urine, blood, serum, other bodily fluids and/or secretions.

Also provided herein is use of an aptamer described herein for detecting SARS-CoV-2 spike protein.

This disclosure provides the first evidence that aptamers can be generated with high affinity to multiple variants of a single protein, including emerging variants, making it well-suited for molecular recognition of rapidly evolving targets such as those found in SARS-CoV-2.

Other features and advantages of the present disclosure will become apparent from the following detailed description. It should be understood, however, that the detailed description and the specific examples, while indicating embodiments of the disclosure, are given by way of illustration only and the scope of the claims should not be limited by these embodiments, but should be given the broadest interpretation consistent with the description as a whole.

### DRAWINGS

Certain embodiments of the disclosure will now be described in greater detail with reference to the attached drawings in which:

FIG. 1A shows the dot blot results of MSA1 for binding to the full spike proteins of the original (WHS) and four variants of SARS-CoV-2 in exemplary embodiments of the disclosure. BA and UA: bound and unbound aptamers.

FIG. 1B shows the dot blot results of MSA5 for binding to the full spike proteins of the original (WHS) and four variants of SARS-CoV-2 in exemplary embodiments of the disclosure. BA and UA: bound and unbound aptamers.

FIG. 2A shows assessment of binding affinity for previously reported DNA aptamers using dot blot assays in exemplary embodiments of the disclosure. FIG. 2A shows binding curve of MSA1 for the spike protein of the original SARS-CoV-2 (WHS) and four variant spike proteins (B.1.1.7S, B.1.351S, P.1S, B.1.429S).

FIG. 2B shows assessment of binding affinity for previously reported DNA aptamers using dot blot assays in exemplary embodiments of the disclosure. FIG. 2B shows binding curve of MSA5 for the spike protein of the original SARS-CoV-2 (WHS) and four variant spike proteins (B.1.1.7S, B.1.351S, P.1S, B.1.429S).

FIG. 3A shows discovery of MSA52 in exemplary embodiments of the disclosure. FIG. 3A shows the sequence of the DNA library used for the original SELEX experiment.

FIG. 3B shows discovery of MSA52 in exemplary embodiments of the disclosure. FIG. 3B shows schematic of reselection used for the discovery of MSA52.

FIG. 3C shows discovery of MSA52 in exemplary embodiments of the disclosure. FIG. 3C shows top 10 members of the MSA52 aptamer family: MSA52R1 (SEQ ID NO: 1), MSA52R2 (SEQ ID NO: 2), MSA52R3 (SEQ ID NO: 3), MSA52R4 (SEQ ID NO: 4), MSA52R5 (SEQ ID NO: 5), MSA52R6 (SEQ ID NO: 6), MSA2R7 (SEQ ID NO: 7), MSA52R8 (SEQ ID NO: 8), MSA52R9 (SEQ ID NO: 9), and MSA52R10 (SEQ ID NO: 10). Each point mutation in relation to the top ranked sequence is highlighted.

FIG. 3D shows discovery of MSA52 in exemplary embodiments of the disclosure. FIG. 3D shows ranking increase of each of the top 10 sequences in the UK (B.1.1.7), BZ (P.1), SA (B.1.351) and CA (B.1.429) pools in comparison to their rankings in the WH pool.

FIG. 4 shows log(frequency) difference plot of the top 15 MSA52 cluster members in WHS, UKS, BZS, SAS and CAS pools as compared to the Round 13 pool in exemplary embodiments of the disclosure.

FIG. 5A shows assessment of the binding affinity of MSA52 for trimeric spike proteins of SARS-CoV-2 and its B.1.1.7, B.1.351, P.1, B.1.429 B.1.617.1, B.1.617.2 and B.1.1.529 variants using dot blot assays in exemplary embodiments of the disclosure. FIG. 5A shows representative dot blot results showing binding of MSA52 to the 8 trimeric spike proteins.

FIG. 5B shows assessment of the binding affinity of MSA52 for trimeric spike proteins of SARS-CoV-2 and its B.1.1.7, B.1.351, P.1, B.1.429 B.1.617.1, B.1.617.2 and B.1.1.529 variants using dot blot assays in exemplary embodiments of the disclosure. FIG. 5B shows binding curves used to derive the K_{d} values for MSA52 for the trimeric spike protein.

FIG. 6 shows competing assays to probe the binding site on the S1 protein of SARS-CoV-2 by MSA52, MSA1 and MSA5 in exemplary embodiments of the disclosure.

FIG. 7A shows the dot blot results for competition assays in exemplary embodiments of the disclosure. FIG. 7A shows MSA1 competed by MSA52. The competed aptamers were at 2.5 nM and radiolabelled (with asterisk); the competing aptamers were unlabelled and had concentrations of 1.2-160 nM. 5 nM of S1 proteins of SARS-CoV-2 were used in the assays. BA and UA: bound and unbound aptamers.

FIG. 7B shows the dot blot results for competition assays in exemplary embodiments of the disclosure. FIG. 7B shows MSA5 competed by MSA52. The competed aptamers were at 2.5 nM and radiolabelled (with asterisk); the competing aptamers were unlabelled and had concentrations of 1.2-160 nM. 5 nM of S1 proteins of SARS-CoV-2 were used in the assays. BA and UA: bound and unbound aptamers.

FIG. 7C shows the dot blot results for competition assays in exemplary embodiments of the disclosure. FIG. 7C shows MSA52 competed by MSA1. The competed aptamers were at 2.5 nM and radiolabelled (with asterisk); the competing aptamers were unlabelled and had concentrations of 1.2-160 nM. 5 nM of S1 proteins of SARS-CoV-2 were used in the assays. BA and UA: bound and unbound aptamers.

FIG. 7D shows the dot blot results for competition assays in exemplary embodiments of the disclosure. FIG. 7D shows MSA52 competed by MSA5. The competed aptamers were at 2.5 nM and radiolabelled (with asterisk); the competing aptamers were unlabelled and had concentrations of 1.2-160 nM. 5 nM of S1 proteins of SARS-CoV-2 were used in the assays. BA and UA: bound and unbound aptamers.

FIG. 7E shows the dot blot results for competition assays in exemplary embodiments of the disclosure. FIG. 7E shows a negative control of MSA52 competed by a mutant sequence (shuffled MSA52, i.e. Mutant Ctrl). The competed aptamers were at 2.5 nM and radiolabelled (with asterisk); the competing aptamers were unlabelled and had concentrations of 1.2-160 nM. 5 nM of S1 proteins of SARS-CoV-2 were used in the assays. BA and UA: bound and unbound aptamers.

FIG. 7F shows the dot blot results for competition assays in exemplary embodiments of the disclosure. FIG. 7F shows self-competitions of MSA52. The competed aptamers were at 2.5 nM and radiolabelled (with asterisk); the competing aptamers were unlabelled and had concentrations of 1.2-160 nM. 5 nM of S1 proteins of SARS-CoV-2 were used in the assays. BA and UA: bound and unbound aptamers.

FIG. 7G shows the dot blot results for competition assays in exemplary embodiments of the disclosure. FIG. 7G shows self-competitions of MSA1. The competed aptamers were at 2.5 nM and radiolabelled (with asterisk); the competing aptamers were unlabelled and had concentrations of 1.2-160 nM. 5 nM of S1 proteins of SARS-CoV-2 were used in the assays. BA and UA: bound and unbound aptamers.

FIG. 7H shows the dot blot results for competition assays in exemplary embodiments of the disclosure. FIG. 7H shows self-competitions of MSA5. The competed aptamers were at 2.5 nM and radiolabelled (with asterisk); the competing aptamers were unlabelled and had concentrations of 1.2-160 nM. 5 nM of S1 proteins of SARS-CoV-2 were used in the assays. BA and UA: bound and unbound aptamers.

FIG. 8 shows specificity tests of MSA52 binding to the spike protein of SARS-CoV-2 and eight control proteins including BSA, human-α-thrombin, and the RBD and spike (S) proteins of SARS-CoV1, RBD proteins of MERS and three seasonal coronavirus 229E, NL63, OC43 in exemplary embodiments of the disclosure. 50 nM proteins were used in the assays.

FIG. 9A shows dot blot results of MSA52 binding to spike protein of SARS-CoV-1 in exemplary embodiments of the disclosure. BA: bound aptamer. UA: unbound aptamer.

FIG. 9B shows dot blot results of MSA52 binding to RBD protein of seasonal coronavirus 229E in exemplary embodiments of the disclosure. BA: bound aptamer. UA: unbound aptamer.

FIG. 9C shows dot blot results of MSA52 binding to RBD protein of seasonal coronavirus OC43 in exemplary embodiments of the disclosure. BA: bound aptamer. UA: unbound aptamer.

FIG. 9D shows affinity (K_{d}) determinations from dot blot results of FIGS. 9A, 9B, and 9C in exemplary embodiments of the disclosure. BA: bound aptamer. UA: unbound aptamer.

FIG. 10A shows dot blot results of MSA52 binding to the pseudotyped virus of the original (WH) SARS-CoV-2 in exemplary embodiments of the disclosure. BA and UA: bound and unbound aptamers.

FIG. 10B shows dot blot results of MSA52 binding to the pseudotyped virus of B.1.1.7 variant of SARS-CoV-2 in exemplary embodiments of the disclosure. BA and UA: bound and unbound aptamers.

FIG. 10C shows dot blot results of MSA52 binding to the pseudotyped virus of B.1.351 variant of SARS-CoV-2 in exemplary embodiments of the disclosure. BA and UA: bound and unbound aptamers.

FIG. 10D shows dot blot results of MSA52 binding to the pseudotyped virus of P.1 variant of SARS-CoV-2 in exemplary embodiments of the disclosure. BA and UA: bound and unbound aptamers.

FIG. 10E shows dot blot results of MSA52 binding to the pseudotyped virus of B.1.429 variant of SARS-CoV-2 in exemplary embodiments of the disclosure. BA and UA: bound and unbound aptamers.

FIG. 10F shows dot blot results of MSA52 binding to the pseudotyped virus of B.1.617.1 variant of SARS-CoV-2 in exemplary embodiments of the disclosure. BA and UA: bound and unbound aptamers.

FIG. 10G shows dot blot results of MSA52 binding to the pseudotyped virus of B.1.617.2 variant of SARS-CoV-2 in exemplary embodiments of the disclosure. BA and UA: bound and unbound aptamers.

FIG. 10H shows dot blot results of MSA52 binding to the pseudotyped virus of B.1.617.2.1 variant of SARS-CoV-2 in exemplary embodiments of the disclosure. BA and UA: bound and unbound aptamers.

FIG. 10I shows dot blot results of binding of MSA52 with control lentiviruses (CV) exemplary embodiments of the disclosure. BA and UA: bound and unbound aptamers.

FIG. 10J shows dot blot results of binding of the inactive mutant sequence of MSA52 (MC) with the original (WH) SARS-CoV-2 in exemplary embodiments of the disclosure. BA and UA: bound and unbound aptamers.

FIG. 11A shows characterization of MSA52 and MSA52-T5 in exemplary embodiments of the disclosure. FIG. 11A shows assessment of binding affinity of MSA52 for lentiviruses pseudotyped to express the spike proteins of the original SARS-CoV-2 virus (WH) as well as B.1.1.7, B.1.351, P.1, B.1,429, B.1.617.1, B.1.617.2, and B.1.617.2.1 variants. MC: Mutant Ctrl.

FIG. 11B shows characterization of MSA52 and MSA52-T5 in exemplary embodiments of the disclosure. FIG. 11B proposed secondary structure of full-length MSA52 (SEQ ID NO: 112).

FIG. 11C shows characterization of MSA52 and MSA52-T5 in exemplary embodiments of the disclosure. FIG. 11B shows proposed secondary structure of a minimized version of MSA52 (named MSA52-T5; SEQ ID NO: 117).

FIG. 12 shows examination of the predicted secondary structure of MSA52 via sequence truncation in exemplary embodiments of the disclosure. Full-length MSA52 is SEQ ID NO: 112. Five truncation mutants were named MSA52-T1 to MSA52-T5 (SEQ ID NOS: 113-117). K_{d} values represent the binding activity for SARS-CoV-2 S1 protein.

FIG. 13A shows schematic illustration of the working principle of sandwich assay of 11 aptamers for the detection of pseudotyped lentiviruses expressing the original SARS-CoV-2 (WH), and the B.1.1.7, B.1.351, P.1 and B.1.617.2 variants in exemplary embodiments of the disclosure.

FIG. 13B shows the original image of a representative 96-well microtiter plate assay of sandwich assay of 11 aptamers for the detection of pseudotyped lentiviruses expressing the original SARS-CoV-2 (WH), and the B.1.1.7, B.1.351, P.1 and B.1.617.2 variants in exemplary embodiments of the disclosure.

FIG. 14A shows sandwich assay of 11 aptamers for the detection of pseudotyped lentiviruses expressing the original SARS-CoV-2 (WH), and the B.1.1.7, B.1.351, P.1 and B.1.617.2 variants in exemplary embodiments of the disclosure. FIG. 14A shows absorbance at 450 nm produced by HRP tagged aptamers in oxidation of TMB in the presence of H₂O₂, followed by quenching with H₂SO₄.

FIG. 14B shows sandwich assay of 11 aptamers for the detection of pseudotyped lentiviruses expressing the original SARS-CoV-2 (WH), and the B.1.1.7, B.1.351, P.1 and B.1.617.2 variants in exemplary embodiments of the disclosure. FIG. 14B shows the image of a representative 96-well microtiter plate assay (the image was processed with Photoshop for enhanced visual effect; the original image is provided in FIG. 13).

FIG. 15A shows the image of a representative 96-well microtiter plate assay of sandwich assay of 11 aptamers for the detection of trimeric spike protein (5 nM) of B.1.1.529 (Omicron) variant of SARS-CoV-2 in exemplary embodiments of the disclosure.

FIG. 15B shows sandwich assay of 11 aptamers for the detection of trimeric spike protein (5 nM) of B.1.1.529 (Omicron) variant of SARS-CoV-2 in exemplary embodiments of the disclosure. FIG. 15B shows absorbance at 450 nm produced by HRP tagged aptamers in oxidation of TMB in the presence of H₂O₂, followed by quenching with H₂SO₄. The A₄₅₀ value of each reaction mixture was corrected for background by subtracting A₄₅₀ observed for the matching BSA reaction mixture.

### DETAILED DESCRIPTION

### I. Definitions

Unless otherwise indicated, the definitions and embodiments described in this and other sections are intended to be applicable to all embodiments and aspects of the present disclosure herein described for which they are suitable as would be understood by a person skilled in the art. It is also to be understood that the terminology used herein is for the purpose of describing particular aspects only and is not intended to be limiting.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies. In addition, all ranges given herein include the end of the ranges and also any intermediate range points, whether explicitly stated or not.

As used in this disclosure, the singular forms “a”, “an” and “the” include plural references unless the content clearly dictates otherwise.

In embodiments comprising an “additional” or “second” component, the second component as used herein is chemically different from the other components or first component. A “third” component is different from the other, first, and second components, and further enumerated or “additional” components are similarly different.

The term “and/or” as used herein means that the listed items are present, or used, individually or in combination. In effect, this term means that “at least one of” or “one or more” of the listed items is used or present.

The abbreviation, “e.g.” is derived from the Latin exempli gratia and is used herein to indicate a non-limiting example. Thus, the abbreviation “e.g.” is synonymous with the term “for example.” The word “or” is intended to include “and” unless the context clearly indicates otherwise.

The term “target”, “analyte” or “target analyte” as used herein refers to any agent, including, but not limited to, a small inorganic molecule, small organic molecule, metal ion, biomolecule, toxin, biopolymer (such as a nucleic acid, carbohydrate, lipid, peptide, protein), cell, tissue, microorganism and virus, for which one would like to sense or detect. The analyte can be either isolated from a natural source or synthetic. The analyte can be a single compound or a class of compounds, such as a class of compounds that share structural or functional features. The term analyte also includes combinations (e.g. mixtures) of compounds or agents such as, but not limited, to combinatorial libraries and samples from an organism or a natural environment.

The term “sample” or “test sample” as used herein refers to any material in which the presence or amount of a target analyte is unknown and can be determined in an assay. The sample can be from any source, for example, any biological (e.g. human or animal samples, including clinical samples), environmental (e.g. water, soil or air) or natural (e.g. plants) source, or from any manufactured or synthetic source (e.g. food or drinks). The sample can be comprised or is suspected of comprising one or more analytes. The sample can be a “biological sample” comprising cellular and non-cellular material, including, but not limited to, tissue samples, saliva, sputum, urine, blood, serum, other bodily fluids and/or secretions. In some embodiments, the sample comprises saliva, sputum, oropharyngeal and/or nasopharyngeal secretions. In some embodiments, the sample comprises saliva.

The term “subject” as used herein includes all members of the animal kingdom including mammals such as a mouse, a rat, a dog and a human.

The term “virus” as used herein refers to an organism of simple structure, composed of proteins and nucleic acids, and capable of reproducing only within specific living cells, using its metabolism. In some embodiments, the virus is an enveloped virus, a non-enveloped virus, a DNA virus, a single-stranded RNA virus and/or a double-stranded RNA virus. Non-limiting examples of virus include rhinovirus, myxovirus (including influenza virus), paramyxovirus, coronavirus such as SARS-CoV-2, norovirus, rotavirus, herpes simplex virus, pox virus (including variola virus), reovirus, adenovirus, enterovirus, encephalomyocarditis virus, cytomegalovirus, varicella zoster virus, rabies lyssavirus and retrovirus (including HIV).

The term “severe acute respiratory syndrome coronavirus 2”, “coronavirus 2”, or “SARS-CoV-2” as used herein refers to a coronavirus first identified in Wuhan, China in 2019 that can cause coronavirus disease (COVID-19). The term includes any variant of the SARS-CoV-2 virus with a variant and/or mutated nucleic acid sequence from the original version identified in Wuhan. Variants include, but are not limited to, UK B.1.1.7 (501Y.V1), South Africa B.1.351 (501Y.V2), Brazil P.1 (501Y.V3), India B.1.617, and B.1.1.529 (Omicron).

The term “nucleic acid” as used herein refers to a biopolymer comprising monomers of nucleotides, such as deoxyribonucleic acid (DNA), ribonucleic acid (RNA) and other polynucleotides of modified nucleotides and/or nucleotide derivatives, and can be either double stranded (ds) or single stranded (ss). In some embodiments, modified nucleotides can contain one or more modified bases (e.g. unusual bases such as inosine, and functional modifications to the bases such as amino), modified backbones (e.g. peptide nucleic acid, PNA) and/or other chemically, enzymatically, or metabolically modified forms.

The term “aptamer” as used herein refers to a short, chemically synthesized nucleic acid molecule or oligonucleotide sequence which can be generated by in vitro selection to fold into specific three-dimensional structures that bind to a specific analyte with dissociation constants, for example, in the pico- to nano-molar range. Aptamers can be single-stranded DNA, and can include RNA, modified nucleotides and/or nucleotide derivatives. Aptamers can also be naturally occurring RNA aptamers termed “riboswitches”. Functional aptamer sequences can also be rationally designed, truncated, conjugated or otherwise modified from original parent (or full length) sequences.

The term “variant” as used herein refers to a subtype of a microorganism that has a genome containing one or more mutations from another strain, for example, a main strain. A variant can be a main strain itself relative to a mutated genome. As such, when referring to two variants, it can be any two subtypes. In relation to SARS-CoV-2 virus, a variant has mutated nucleic acid sequence from the original version identified in Wuhan, and it includes, but is not limited to, B.1.1.7, B.1.351, P.1, B.1.617, and B.1.1.529, and the Wuhan strain itself is considered a variant to the other subtypes. A variant can have potential consequences including increased transmissibility, increased morbidity, increased mortality, ability to evade detection by diagnostic tests, decreased susceptibility to antiviral drugs, decreased susceptibility to neutralizing antibodies, ability to evade natural immunity, ability to infect vaccinated individuals, increased risk of particular conditions such as multisystem inflammatory syndrome or long COVID, and/or increased affinity for particular demographic or clinical groups, such as children or immunocompromised individuals. In relation to any particular molecular target such as a protein, a variant can be, for example, a mutated protein having at least 75% sequence identity with the original protein, and/or a functional variant retaining some functional aspects of the original protein. A variant can be a main, wildtype, or original target analyte (e.g. strain or protein) itself relative to a mutated target analyte. As such, when referring to two target analyte variants, it can be any two subtypes of a target analyte. A skilled person is able to recognize target analyte variants whether at the level of, for example, microorganism or protein.

The term “systematic evolution of ligands by exponential enrichment” or “SELEX” as used herein refers to a method for the selection of an aptamer that specifically binds to a target molecule, for example as described in Ellington, A. D. & Szostak, J. W., “In vitro selection of RNA molecules that bind specific ligands.” Nature 346, 818-822 (1990), McConnell, E. M. et al., “Biosensors Made of Synthetic Functional Nucleic Acids Toward Better Human Health.” Anal. Chem. 92, 327-344 (2020), U.S. Pat. Nos. 5,475,096, and 5,270,163, each of which is herein incorporated by reference in its entirety. SELEX involves immobilizing a selected target on a column and a solution containing a library or assortment of random DNA or RNA molecules is contacted with the target under conditions that are favorable for binding. After a certain amount of time the column is flushed, so that unbound nucleic acid molecules are washed from the column and those nucleotide molecules which bind to the target remain on the column. The nucleic acid-target complexes are then dissociated and the nucleic acid molecules that were selected by binding to the target are amplified. The cycle can be repeated to achieve a higher affinity nucleic acid. A SELEX-compatible aptamer pool have aptamers that are fully or partially randomized oligonucleotide sequences of some length flanked by defined regions which allow PCR amplification of those randomized regions (or in vitro transcription if it is RNA SELEX). For example, a SELEX-compatible aptamer pool can have a plurality of nucleic acid molecules comprising at least one random nucleotide domain that has an aptamer-like structure, the random domain is flanked by a 5′-end region and a 3′-end region, and the 5′-end region can hybridize to the 3′-end region to form a duplex DNA element. The random domain can have a length of about 40 nucleotides, and as such, an example of a SELEX-compatible aptamer pool comprises a DNA library having the nucleotide sequence of SEQ ID NO: 101.

### II. Methods, Uses, and Aptamers

Disclosed herein is the method for identifying or producing DNA aptamers against variants of SARS-CoV-2 spike protein subunit S1 from a pre-structured random DNA library using parallel systematic evolution of ligands by exponential enrichment (SELEX), high-throughput sequencing and sequence alignment analysis. Inventors show that aptamers can be generated with high affinity to multiple variants of a single protein, making it well-suited for molecular recognition of rapidly evolving targets such as those found in SARS-CoV-2.

Accordingly, herein provided is a method of identifying or producing an aptamer capable of binding to at least two target analyte variants, the method comprising:
a) generating a mixture of systematic evolution of ligands by exponential enrichment (SELEX)-selected aptamers by at least one-round of SELEX with a SELEX-compatible aptamer pool against the at least two target analyte variants in parallel or sequentially;
b) sequencing the mixture of SELEX-selected aptamers that form complexes with each of the at least two target analyte variants by high-throughput sequencing;
c) aligning the mixture of SELEX-selected aptamers that form complexes with each of the at least two target analyte variants by sequence alignment analysis; and
d) identifying or producing the aptamer capable of binding to the at least two target analyte variants.

In some embodiments, the method comprises, prior to a), generating an aptamer pool compatible with SELEX. In some embodiments, the SELEX-compatible aptamer pool comprises a plurality of nucleic acid molecules comprising at least one random nucleotide domain having an aptamer-like structure, the random nucleotide domain flanked by a 5′-end region and a 3′-end region. In some embodiments, the 5′-end region hybridizes to the 3′-end region to form a duplex DNA element. In some embodiments, the 5′-end region comprises a nucleotide sequence of SEQ ID NO: 102 or 103, and the 3′-end region comprises a nucleotide sequence of SEQ ID NO: 132 or the reverse complement of SEQ ID NO: 104 or 105. In some embodiments, the SELEX-compatible aptamer pool comprises a plurality of nucleic acid molecules having the nucleotide sequence of SEQ ID NO: 101. In some embodiments, the SELEX-compatible aptamer pool comprises at least one aptamer comprising a nucleotide sequence selected from the group consisting of SEQ ID NOS: 108, 112-117, a functional fragment, and functional variant thereof, and/or comprises at least one aptamer comprising a 5′-end region, a 3′-end region, and a nucleotide sequence selected from the group consisting of SEQ ID NOS: 1-100, a functional fragment thereof, and a functional variant thereof.

In some embodiments, SELEX comprises contacting a pool of aptamers with the target analyte variant under conditions favorable for binding, partitioning unbound aptamers from those aptamers which have bound to the target analyte variant, dissociating the aptamer-target analyte variant complex, amplifying the aptamer dissociated from the aptamer-target analyte variant to yield a target analyte variant-enriched mixture of aptamers. In some embodiments, the target analyte variant is a microorganism, a virus, and/or a molecule present in a microorganism or a virus. In some embodiments, the target analyte variant is a protein. In some embodiments, the target analyte variant is a protein from SARS-CoV-2. In some embodiments, the target analyte variant is SARS-CoV-2 spike protein.

In some embodiments, the method comprises in a) generating a mixture of SELEX-selected aptamers by at least three, four, five, six, seven, eight, or nine target analyte variants. In some embodiments, the method comprises in a) generating a mixture of SELEX-selected aptamers by 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 target analyte variants. In some embodiments, the method comprises in a) 1, 2, 3, 4, 5, 6, 7, 8, 9, 10, 11, 12, 13, 14, 15, 16, 17, 18, 19, or 20 rounds of SELEX with a SELEX-compatible aptamer pool.

In some embodiments, the high-throughput sequencing comprises single-molecule real-time sequencing, ion semiconductor sequencing, pyrosequencing, sequencing by synthesis, combinatorial probe anchor synthesis sequencing, sequencing by ligation, nanopore sequencing, or GenapSys™ sequencing. In some embodiments, the high-throughput sequencing comprises sequencing by synthesis.

In some embodiments, the method comprises at least one of merging of sequences into a consensus sense read, dereplicating, clustering at 90% identity, multiple sequence alignments, converting to sequence logos, identifying sequence copy number, identifying sequence frequency, and identifying cluster linkage.

In some embodiments, the method comprises a step or method described in the Examples.

In another aspect, there is provided an DNA aptamer, denoted MSA52, that displays universally high affinity for the spike proteins of wildtype SARS-CoV-2 as well as the Alpha, Beta, Gamma, Epsilon, Kappa Delta and Omicron variants. Using an aptamer pool produced from round 13 of selection against the S1 domain of the wildtype spike protein, one-round SELEX experiments were carried out using five different trimeric spike proteins from variants, followed by high-throughput sequencing and sequence alignment analysis of aptamers that formed complexes with all proteins. MSA52 showed K_{d} values ranging from 2-10 nM for all variant spike proteins, and also bound similarly to variants not present in the reselection experiments. This aptamer also recognized pseudotyped lentiviruses (PL) expressing eight different spike proteins of SARS-CoV-2 with Kd values between 20-50 pM.

In some embodiments, the aptamer comprises a 5′-end region, a 3′-end region, and a nucleotide sequence selected from the group consisting of SEQ ID NOS: 1-100, a functional fragment, and functional variant thereof, or comprises a nucleotide sequence selected from the group consisting of SEQ ID NOS: 108, 112-117, a functional fragment, and functional variant thereof. In some embodiments, the aptamer comprises a 5′-end region, a 3′-end region, and a nucleotide sequence selected from the group consisting of SEQ ID NOS: 2-100, a functional fragment, and functional variant thereof, or comprises a nucleotide sequence selected from the group consisting of SEQ ID NOS: 113-117, a functional fragment, and functional variant thereof. In some embodiments, an aptamer is provided comprising a nucleotide sequence selected from the group consisting of SEQ ID NOS: 106-117, a functional fragment, and functional variant thereof. In some embodiments, the 5′-end region comprises a nucleotide sequence of SEQ ID NO: 102 or 103, and the 3′-end region comprises a nucleotide sequence of SEQ ID NO: 132 or the reverse complement of SEQ ID NO: 104 or 105. In some embodiments, the aptamer comprises a 5′-end region, a 3′-end region, and a nucleotide sequence selected from the group consisting of SEQ ID NOS: 2-10, a functional fragment, and functional variant thereof. In some embodiments, the aptamer comprises a nucleotide sequence comprising SEQ ID NO: 116 or 117.

Also provided is a method for detecting the presence of SARS-CoV-2 spike protein in a sample, the method comprising:
a) contacting the sample with an aptamer described herein, wherein the aptamer binds the spike protein; and
b) detecting the presence of the aptamer bound to the spike protein in the sample, wherein detection of bound aptamer indicates the presence of SARS-CoV-2 spike protein or variant thereof.

In some embodiments, the SARS-CoV-2 spike protein variant comprises B.1.1.7, B.1.351, P.1, B.1.429, B.1.617.1 B.1.617.2, B.1.617.2.1, or B.1.1.529 spike protein variant. In some embodiments, the aptamer comprises a 5′-end region, a 3′-end region, and a nucleotide sequence selected from the group consisting of SEQ ID NOS: 1-100, a functional fragment, and functional variant thereof, or comprises a nucleotide sequence selected from the group consisting of SEQ ID NOS: 106-117, a functional fragment, and functional variant thereof. In some embodiments, the aptamer comprises a 5′-end region, a 3′-end region, and a nucleotide sequence selected from the group consisting of SEQ ID NOS: 1-10, a functional fragment, and functional variant thereof. In some embodiments, the aptamer comprises a 5′-end region, a 3′-end region, and a nucleotide sequence of SEQ ID NO: 1, a functional fragment, and functional variant thereof. In some embodiments, the aptamer comprises a 5′-end region, a 3′-end region, and a nucleotide sequence of SEQ ID NO: 1. In some embodiments, the 5′-end region comprises a nucleotide sequence of SEQ ID NO: 102 or 103, and the 3′-end region comprises a nucleotide sequence of SEQ ID NO: 132 or the reverse complement of SEQ ID NO: 104 or 105. In some embodiments, the aptamer comprises a nucleotide sequence selected from the group consisting of SEQ ID NOS: 108, 112-117, a functional fragment, and functional variant thereof. In some embodiments, the aptamer comprises a nucleotide sequence selected from the group consisting of SEQ ID NOS: 108, 112, 116, 117, a functional fragment, and functional variant thereof. In some embodiments, the aptamer comprises a nucleotide sequence of SEQ ID NO: 108, 112, 116, or 117. In some embodiments, the aptamer comprises a nucleotide sequence of SEQ ID NO: 108, a functional fragment, or functional variant thereof. In some embodiments, the aptamer comprises a nucleotide sequence of SEQ ID NO: 108. In some embodiments, the aptamer comprises a nucleotide sequence of SEQ ID NO: 112, a functional fragment, or functional variant thereof. In some embodiments, the aptamer comprises a nucleotide sequence of SEQ ID NO: 112. In some embodiments, the aptamer comprises a nucleotide sequence of SEQ ID NO: 116, a functional fragment, or functional variant thereof. In some embodiments, the aptamer comprises a nucleotide sequence of SEQ ID NO: 116. In some embodiments, the aptamer comprises a nucleotide sequence of SEQ ID NO: 117, a functional fragment, or functional variant thereof. In some embodiments, the aptamer comprises a nucleotide sequence of SEQ ID NO: 117. In some embodiments, the method detects SARS-CoV-2 infection in a subject. In some embodiments, the sample is saliva, sputum, urine, blood, serum, other bodily fluids and/or secretions.

Also provided is a use of an aptamer described herein for detecting SARS-CoV-2 spike protein.

Also provided is a nucleotide comprising a sequence selected from the group consisting of SEQ ID NOS: 1-132.

It will be understood that any component defined herein as being included can be explicitly excluded by way of proviso or negative limitation, such as any specific compounds or method steps, whether implicitly or explicitly defined herein.

### Examples

The following non-limiting examples are illustrative of the present disclosure:

### Methods

Materials and reagents: DNA oligonucleotides were obtained from Integrated DNA Technologies (IDT) and purified by standard 10% denaturing (8 M urea) polyacrylamide gel electrophoresis (dPAGE) before use. The sequences are listed in Table IA and Table 3. The Wuhan SARS-CoV-2 spike protein subunit S1 (catalog number: 40591-V08B1) was purchased from Sino Biological Inc. The Wuhan SARS-CoV-2 full spike protein (WHS, molecular weight 140 kDa), spike-pseudotyped lentiviruses for Wuhan SARS-CoV-2 (WH), variant B.1.351, variant P.1 and control lentivirus were prepared using standard methods. The full spike proteins for the variants B.1.1.7 (catalog number: SPN-C52H6), B.1.617.1 (SPN-C52Hr), B.1.617.2 (SPN-C52He), and B.1.1.529 (SPN-C52 Hz) were all expressed from human 293 cells (HEK293) and obtained from Acro Biosystems. The spike proteins for the variants B.1.351 (510333-1), P.1 (100989-1) and B.1.429 (101057) were all expressed from human 293 cells (HEK293) and obtained from BPS Biosciences Inc. The spike and RBD proteins for SARS-CoV-1, MERS and seasonal coronavirus 229E, NL63 and OC43 were provided by Dr. Miller's lab at McMaster University. The concentrations of the proteins were quantified using bicinchoninic acid (BCA) protein assay kits from Thermo Scientific (Catalog number: 23225). The SARS-CoV-2 spike-pseudotyped lentivirus for the variant B.1.1.7 (catalog number: 78112-1), B.1.429 (78172-1), B.1.617.1 (78205-1), B.1.617.2 (78216-1) and B.1.617.2.1 (78219-1) were obtained from BPS Bioscience. Nitrocellulose blotting membranes (catalog No. 10600125) were purchased from GE Healthcare Inc. Nylon hybridization transfer membranes (NEF994001PK) were purchased from PerkinElmer Inc. (Woodbridge, ON, Canada). T4 polynucleotide kinase (PNK), adenosine triphosphate (ATP) and deoxyribonucleoside 5′-triphosphates (dNTPs) were purchased from Thermo Scientific (Ottawa, ON, Canada). γ-[³²P]-ATP was acquired from PerkinElmer. Bovine serum albumin (BSA) and human thrombin were purchased from Sigma-Aldrich (Oakville, Canada). 4-(2-hydroxyethyl)-1-piperazineethanesulfonic acid (HEPES), sodium chloride, magnesium chloride, Tween-20 and all other chemicals were purchased from Sigma-Aldrich (Oakville, Canada) and used without further purification. Milli-Q water was used for all the experiments.

Radiolabelling of DNA aptamers: DNA aptamers were labeled with γ-[³²P] ATP at the 5′-end using PNK reactions according to the manufacturer's protocol. Briefly, 2 μL of 1 μM DNA aptamers were mixed with 2 μL of γ-[³²P] ATP, 1 μL of 10×PNK reaction buffer A, 10 U (U: unit) of PNK and 4 μL water. The mixture was incubated at 37° C. for 20 min, and then purified by 10% dPAGE.

Preparation of recombinant full trimeric spike protein: A detailed protocol outlining protein production can be found in Stadlbauer et al. 2020 [33]. The plasmid encoding the mammalian cell codon optimized sequence for Wuhan SARS-CoV-2 full length spike protein was described in Amanat, F. et al. A serological assay to detect SARS-CoV-2 seroconversion in humans. Nat. Med. 26, 1033-1036 (2020) [34]. In brief, proteins were produced in Expi293 cells (ThermoFisher Scientific) using the manufacturers' instructions. When culture viability reached 40%, supernatants were collected and spun at 500 g for 5 minutes. The supernatant was then incubated with 1 ml of Ni-NTA agarose (Qiagen) per 25 ml of transfected cell supernatant overnight, with shaking, at 4° C. The following day 10 ml polypropylene gravity flow columns (Qiagen) were used to elute the protein. Spike proteins were concentrated in 50 kDa Amicon centrifugal units (Millipore) prior to being resuspended in phosphate buffered saline (PBS).

Preparation of lentivirus: Wuhan SARS-CoV-2 S protein pseudotyped lentivirus was produced as described by Crawford et al.[35] SARS-Related Coronavirus 2, Wuhan-Hu-1 Spike-Pseudotyped Lentiviral Kit (BEI catalog number NR-52948) was obtained through BEI resources, National Institute of Allergy and Infectious Diseases, National Institutes of Health. In brief, HEK293T cells were seeded in 15 cm dishes at 1.1×10⁷ cells/mL in 15 mL of standard Dulbecco's Modified Eagle Medium (DMEM). 16-24 hours post seeding, cells were co-transfected with HDM-nCoV-Spike-IDTopt-ALAYT (BEI catalog number NR-52515), pHAGE-CMV-Luc2-IRES-ZsGreen-W (BEI catalog number NR-52516), HDM-Hgpm2 (BEI catalog number NR-52517), HDM-tat1b (BEI catalog number NR-52518) and pRC-CMV-Rev1b (BEI catalog NR-52519). 18-24 h post-transfection the media was replaced with full DMEM and 60 h post transfection, the supernatant was collected and filtered with a 0.45 μm filter and stored at −80° C. until future use. For purification, 40 mL of supernatant was concentrated by spinning at 19,400 rpm for 2 h. The resulting pellet was resuspended in 400 μl of HBSS, followed by 15 min of continuous vortexing at room temperature. Protein concentration was confirmed by the BCA assay.

Dot blot binding assays with spike protein: Dot blot assays were performed by using a Whatman Minifold-1 96-well apparatus and a vacuum pump. Before experiments, nitrocellulose membranes and nylon membranes were incubated in 1×SB buffer for 1 h. γ-[³²P] labelled DNA aptamers (1 nM) were dissolved in the binding buffer and heated at 90° C. for 5 min, and then cooled at room temperature for 20 min. Spike proteins were dissolved and diluted in the same buffer. 5 μL of the above aptamer solution was mixed with 15 μL of spike protein with different concentrations. The mixture was incubated at room temperature for 1 h. The dot blot apparatus was assembled with a nitrocellulose membrane on the top, a nylon membrane in the middle and a wetted Whatman paper in the bottom. After washing each well with 100 μL of binding buffer, the binding mixtures were loaded and drained by the vacuum pump (force: 550 mmHg for 8 seconds). The wells were then washed twice with 100 μL binding buffer. The membranes were imaged using a Typhoon 9200 imager (GE Healthcare) and analyzed using Image J software.[28] Each binding assay was performed 3 times. The bound fraction was quantified and plotted against the concentration of the protein. The K_{d} values were derived via curve fitting using Origin 8.0 using the equation Y=BₘₐₓX/(K_{d}+X) (Y is the bound fraction of aptamer with protein, Bₘₐₓ is the maximum bound fraction of aptamer, and X is protein concentration).

Dot Blot Binding Assays with SARS-CoV-2 Spike-Pseudotyped Lentivirus: Dot blot assays with SARS-CoV-2 spike-pseudotyped lentivirus and the control lentivirus without spike protein were performed using the same procedure as described above except: the aptamer solutions were incubated with different concentrations of viruses (0-600 pM of viral particles; corresponding to 0-3×10¹¹ cp mL⁻¹) for 10 min, followed by performing dot blot assays.

Rapid selection of aptamers for the spike proteins of SARS-CoV-2 variants: The one-round selection processes were carried out by native gel-based methods using a pre-enriched DNA pool for the spike protein of wildtype SARS-CoV-2 after 13 rounds of selection.[17] Briefly, the FAM-labelled pre-enriched DNA pool was diluted in 10 μL selection buffer (1×SB; 50 mM HEPES, 6 mM KCl, 150 mM NaCl, 2.5 mM CaCl₂, 2.5 mM MgCl₂, 0.01% v/v Tween-20, pH 7.4) to 100 nM, followed by heating at 90° C. for 5 min and annealing at room temperature for 10 min. Spike proteins (10 μL, 800 nM, in 1×SB) of different SARS-CoV-2 variants were then mixed with the DNA pool and incubated at 23° C. for 30 min. The DNA bound with spike proteins was separated from unbound DNA using 10% (v/v) native PAGE, which was imaged using a Typhoon imaging system (Typhoon™ FLA 9500, GE Healthcare, USA). Afterward, the bound DNA was cut and eluted from the gel by incubating in 1×Taq buffer (200 μL, 50 mM KCl, 10 mM Tris-HCl, 1.5 mM MgCl₂, 1% v/v Triton X-100, pH 9.0) at 23° C. for 20 min. The DNA in the supernatant was amplified by PCR, after the addition of FP1 (10 μL, 10 μM), RP1 (10 μL, 10 μM), Taq DNA polymerase (2 μL, 5 U/μL), and dNTPs (20 μL, 2 mM). The PCR temperature profile was set as follows: preheating at 94° C. for 30 s; temperature cycles of 94° C. for 30 s, 50° C. for 30 s, and 72° C. for 30 s; annealing at 72° C. for 5 min. The PCR products were then further amplified by PCR using sequencing primers and then analyzed using the MiSeq (Illumina) sequencing platform. The selection processes were simultaneously carried out for WHS, UKS, SAS, BZS and CAS.

Sequencing data analysis: Sequencing samples were prepared from each parallel SELEX experiment by PCR tagging with Illumina sequencing primers. Samples were size purified by agarose gel electrophoresis prior to being quantified by measuring absorbance at 260 nm. Tagged samples were pooled and paired-end sequenced on an Illumina MiSeq high-throughput DNA sequencer. Sequence data processing was performed on a Windows 10 computer running Ubuntu 20.04 under WSL2. Raw paired-end reads were trimmed of sequencing and library primers using cutadapt 3.4.[29] Trimmed paired-end reads were then: 1) merged into a consensus sense read; 2) dereplicated; and 3) clustered at 90% identity using USEARCH v11.0.667_i86linux32.[30] Sequence frequencies and ranking lists were generated using custom Python scripts. Multiple sequence alignments were performed using MUSCLE v3.8.1551 and converted to sequence logos using WebLogo 3.7.8.[31,32] Processed sequencing data and cluster linkage data were stored on a MySQL 8.0.22 database. Analysis of sequence copy number, frequency, cluster linkage and data plots were performed using the database and Microsoft Excel.

ELABA for Pseudotyped Lentivirus and the spike protein of B.1.1.529 (Omicron): The ELABA (enzyme-linked aptamer binding assay) for different pseudotyped lentivirus and the B.1.1.529 spike protein (B.1.1.529S) was conducted on a Pierce™ streptavidin-coated microtiter plate (Thermo Scientific™, catalog No. 15121). The plate wells were washed three times with 200 μL 1×SB buffer (50 mM HEPES, 6 mM KCl, 150 mM NaCl, 2.5 mM CaCl₂, 2.5 mM MgCl2, 0.01% v/v Tween-20, pH 7.4) after the binding of each reagent. First, the plate wells were blocked with blocking buffer (300 μL, 1×SB buffer, 10% w/v BSA) by incubation at 37° C. for 1 h. Biotinylated aptamers (100 μL, 200 nM) in dilution buffer (1×SB with 0.1% w/v BSA) were then added to the wells and incubated at 22° C. for 30 min. The pseudotyped lentivirus (100 μL, 2 pM) or B.1.1.529S (100 μL, 5 nM) in dilution buffer was then introduced and captured by aptamers on the plate, followed by incubation at 22° C. for 1 h. Next, the same biotinylated aptamer (100 μL, 200 nM) was added again and used as a reporter aptamer to bind with the virus with incubation at 22° C. for 30 min. Subsequently, streptavidin-HRP (100 μL, 1:2000 dilution, New England Biolabs, catalog No. 3999S) in dilution buffer was introduced to bind to the reporter aptamer with incubation at 22° C. for 30 min. Finally, 1-Step™ Ultra TMB-ELISA Substrate Solution (100 μL, Thermo Scientific™, catalog No. 34028) was introduced and reacted at 22° C. for 20 min. H₂SO₄ (20 μL, 2 M) was used to terminate the catalytic reaction, converting TMB to its oxidized product, which was measured by a plate reader (Tecan, Switzerland) at 450 nm.

### Results and Discussion

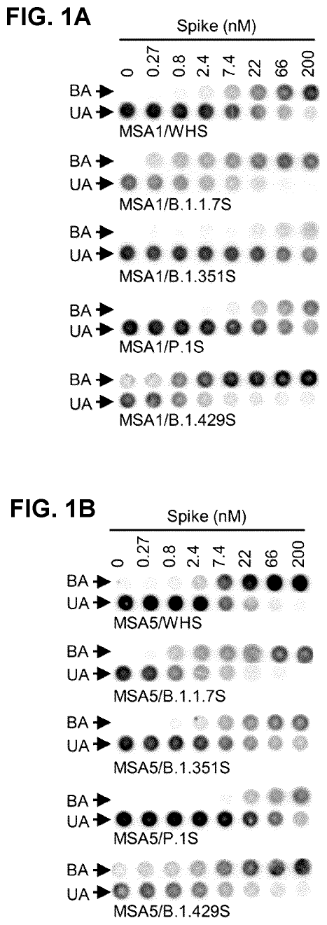

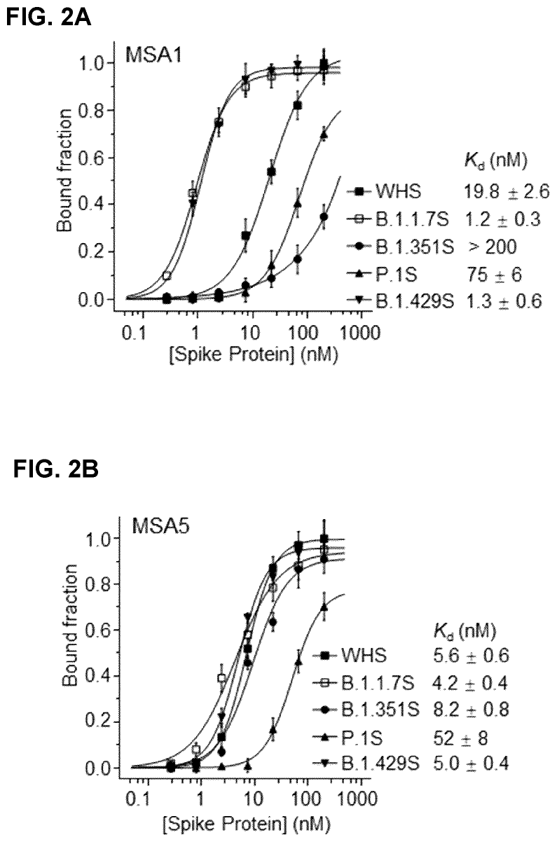

Assessment of binding affinity of previously reported aptamers for SARS-CoV-2 variants: As the starting point for examining aptamers for recognition of variant spike proteins, dot blot assays were used (FIG. 1) to examine the binding affinity of MSA1 (FIG. 2A) and MSA5 (FIG. 2B) for the full trimeric spike proteins of the original SARS-CoV-2 (WHS) and four VoCs: B.1.1.7S (UKS), B.1.429S (CAS), B.1.351S (SAS) and P.1S (BZS; “S” in the names stands for the full spike protein). At the time of these experiments, neither the S1 nor full trimeric spike proteins were available for the Delta and Omicron variants. MSA1 showed strong affinity for both B.1.1.7S (K_{d}=1.2 nM) and B.1.429S (K_{d}=1.3 nM), but had significantly reduced affinity for P.1S (K_{d}=75 nM) and very poor affinity for B.1.351S (K_{d}>200 nM), as compared to the K_{d} of 19.8 nM for the original Wuhan virus. MSA5 showed similar levels of affinity for WHS (K_{d}=5.6 nM), B.1.1.7S (K_{d}=4.2 nM), B.1.429S (K_{d}=5.0 nM), and B.1.351S (K_{d}=8.2 nM). However, it had significantly decreased affinity for P.1S (K_{d}=52 nM).

The results presented above show that the epitopes for MSA1 and MSA5 are not identical, and more importantly, the epitopes recognized by both aptamers are sensitive to the conformational changes of the spike proteins caused by the mutations associated with some of the VoCs. Specifically, MSA5 is sensitive to the conformational changes of P.1S while MSA1 is sensitive to that of both P.1S and B.1.351S. Several other published aptamers were also examined for binding to spike proteins of six different variants of SARS-CoV-2 and none of them were able to recognize WHS, B.1.1.7S, B.1.429S, B.1.351S, P.1S, and B.1.1.529S with uniformly excellent affinity. These findings show that these aptamers cannot function as universal affinity agents targeting all variant spike proteins for either therapeutic or diagnostic applications.

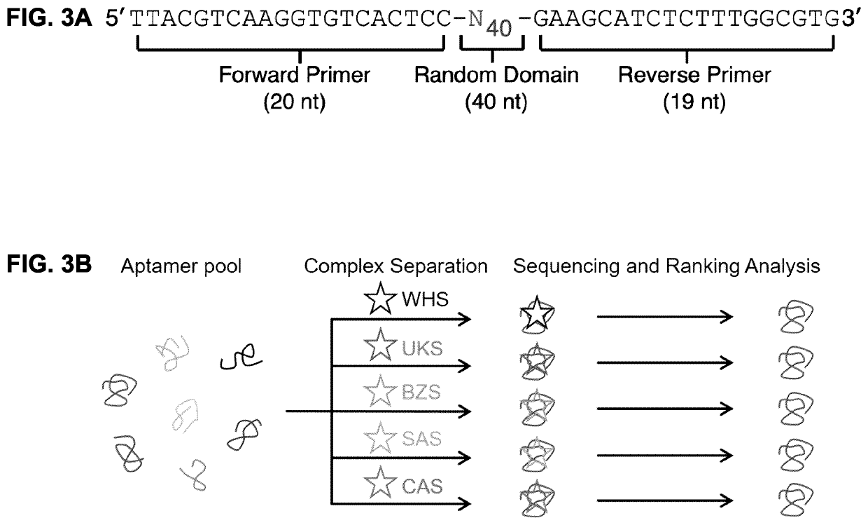

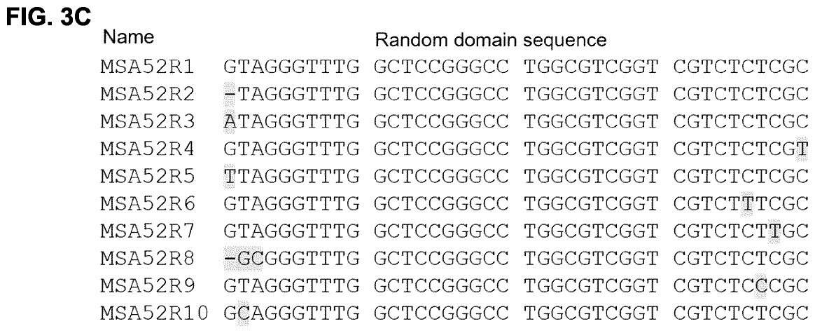

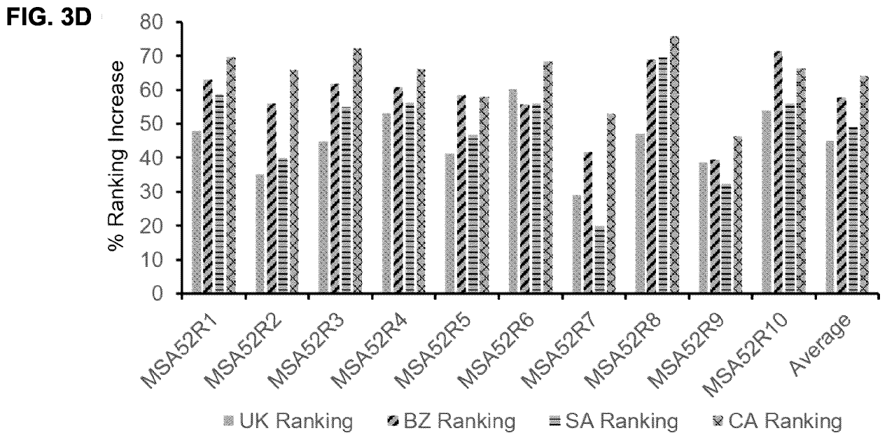

Selection of DNA aptamers for diverse SARS-CoV-2 variants: The recently published aptamers for SARS-CoV-2 were selected using a pre-structured DNA library that placed a 40-nt random region (FIG. 3A) in a hairpin-structured arrangement often observed with many published aptamers, resulting in the isolation of MSA1 and MSA5, the top ranked and 5^{th} ranked aptamer candidates.[17] However, further examination of the binding affinity of other aptamers, including two low-ranked aptamers, MSA50 (the 50^{th} ranked aptamer candidate; K_{d}=10.2 nM) and MSA439 (the 439^{th} ranked candidate; K_{d}=36.9 nM), showed that even lowly ranked candidates in the final pool still exhibited excellent affinity for WHS. Based on these results, it was reasonably considered that the enriched aptamer pool might also contain aptamer candidates that could universally recognize the spike proteins of the currently circulating VoCs and perhaps even emerging VoCs.

To test this idea and to quickly generate aptamer candidates for recognition of the spike proteins of diverse VoCs, five parallel one-round SELEX experiments were carried out with the Generation-13 pool and each of the five spike proteins that were commercially available at the time, including the original SARS-CoV-2 and its B.1.1.7, B.1.351, P.1 and B.1.429 variants (FIG. 3B). The spike protein for the B.1.617.2 Delta and B.1.1.529S Omicron variants were not available when this experiment was carried out. SELEX was done using electrophoretic mobility shift assays (EMSA) wherein each spike protein was first incubated with the DNA pool, followed by separation of the spike protein-DNA complexes from the unbound DNA using native polyacrylamide gel electrophoresis. After elution from the gel, the bound DNA was amplified by PCR The amplified DNA samples were then subjected to high-throughput sequencing analysis.

The ranking of the top 100 aptamer sequences previously discovered (named MSA1 to MSA100) in each pool was then compared and found that the ranking of a particular aptamer, named MSA52, was significantly increased after reselection. MSA52 was ranked #52 in inventors' original selection performed with the S1 subunit of WHS; however, the ranking increased to 46^{th}, 24^{th}, 19^{th}, 17^{th}, and 14th in the pools established respectively with WHS, UKS, BZS, SAS and CAS, showing that MSA52 competed favorably with the top-ranking sequences.

There are a total of 321 members of the MSA52 aptamer family; the sequences, ranking within each pool and frequency values of the top 100 members are provided in Table IA and Table 1B. FIG. 3C lists the top 10 sequences in the family (which are named MSA52R1-10; SEQ ID NOS: 1-10, respectively). The ranking increase of each of the top 10 sequences in the UK, BZ, SA and CA pools was then calculated in comparison to their rankings in the WH pool. Interestingly, all these top 10 sequences exhibit ˜50% ranking increases in each variant pool (FIG. 3D), once again confirming that the members of this aptamer family competed well for binding to all the four variants used for the SELEX experiment.

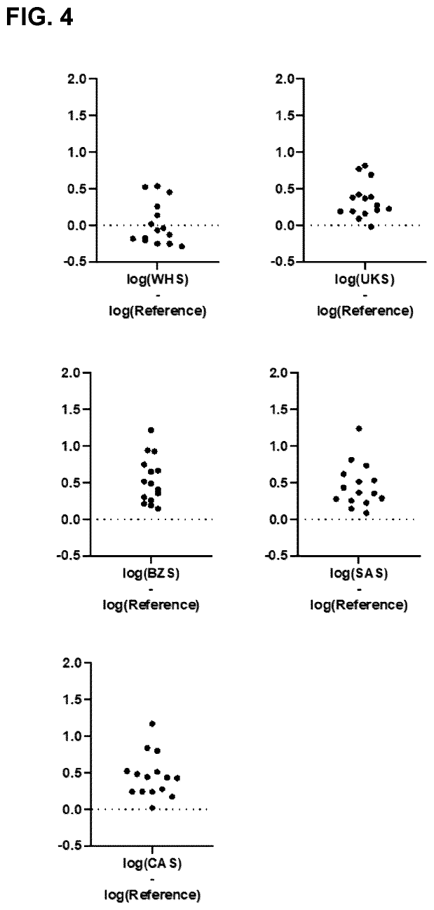

A t-test was performed to compare the ratio of frequencies of the top 15 members of the MSA52 cluster in each of the 5 selections vs. the initial (round 13) frequencies (FIG. 4 and Table 2). These sequences were indeed significantly enriched for the pools selected with variant protein targets (P<0.0001). In contrast, the difference between the round 13 reference population and the WHS pool is insignificant (P=0.7718; the geometric mean of ratio and 95% CI can be found in Table 2). Taken together, the sequencing data analysis show that MSA52 can function as a universal spike protein binding aptamer that is insensitive to the mutations observed in spike proteins of the B.1.1.7, B.1.351, P.1 and B.1.429 variants.

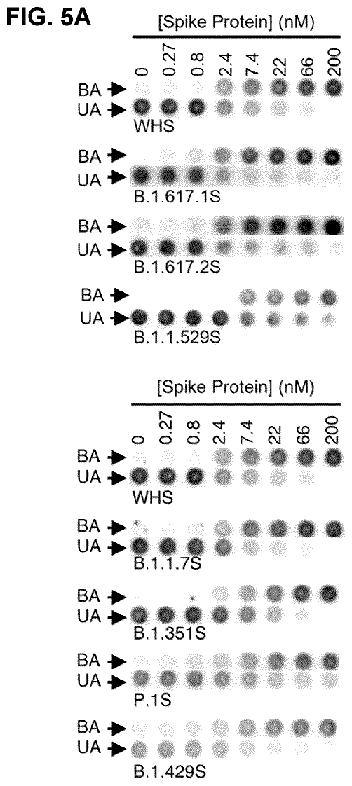

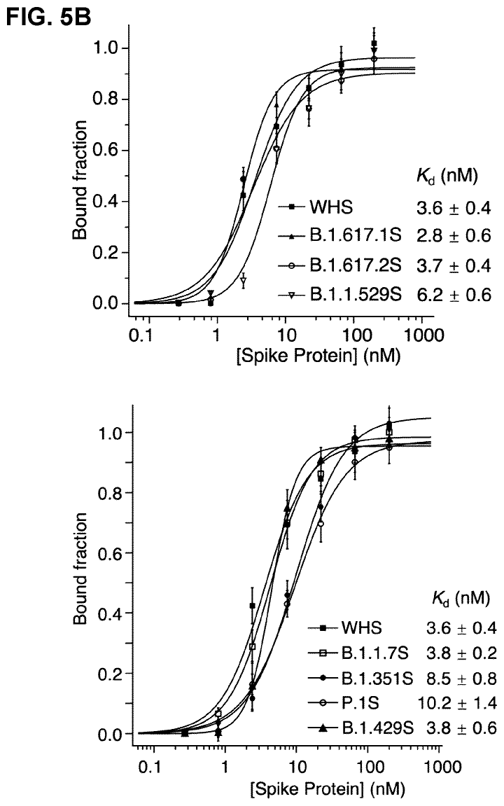

Binding of variant spike proteins by MSA52: The binding affinity of MSA52 (also known as MSA52R1 in FIG. 3C) for the full trimeric spike (TS) proteins of SARS-CoV-2 and its B.1.1.7, B.1.351, P.1, B.1.429 variants was assessed using dot blot assays. Representative dot blots from these experiments are shown in FIG. 5A, and binding curves of bound fraction vs. protein concentration are plotted in FIG. 5B to derive the K_{d} values. As expected, MSA52 showed strong binding to all five TS proteins, the K_{d} values varied between only 3.6-10.2 nM for the five TS variants. The aptamer showed nearly identical affinity for WHS (3.6 nM), UKS (B.1.1.7S; 3.8 nM), and CAS (B.1.429S; 3.8 nM), and slightly reduced but still excellent affinity for SAS (B.1.351S; 8.5 nM) and BZS (P.1S; 10.2 nM). The binding data is consistent with the selection outcome, indicating that the increase of the frequency of MSA52 and its related family members was a result of its high affinity for the spike proteins of the variants.

As noted above, the spike proteins of the newly emerged VoCs were not available when the one-round SELEX experiments were conducted. Since then, the spike proteins of the Kappa (B.1.617.1), Delta (B.1.617.2), and Omicron (B.1.1.529) variants became available, providing the opportunity to assess whether the MSA52 aptamer could detect emerging variants not used for the selection experiment. Using dot blot assays (FIG. 5A), the binding of MSA52 to the TS proteins of these three new variants (named B.1.617.1S, B.1.617.2S and B.1.1.529S) was tested. Binding curves (FIG. 5B) showed that the aptamer binds to B.1.617.1S, B.1.617.2S and B.1.1.529S with K_{d} values of 2.8 nM, 3.7 nM and 3.7 nM and 6.2 nM, respectively, which were nearly identical to the K_{d} value observed for WHS (K_{d}=3.6 nM). This observation further confirms MSA52 is a “universal” aptamer for spike protein recognition, as it can recognize all 7 spike protein variants of SARS-CoV-2 inventors have tested so far, including those not included during the one-round reselection experiment.

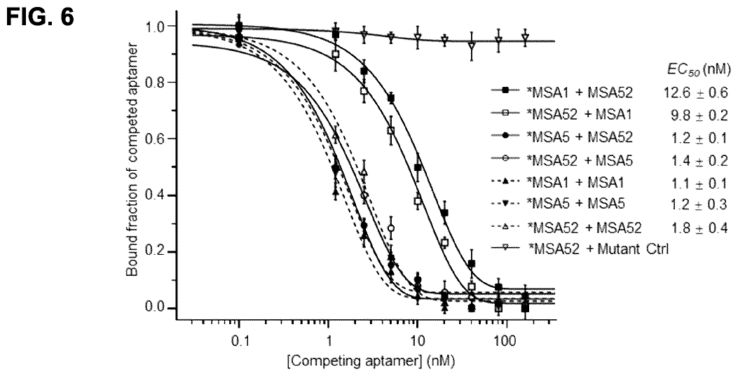

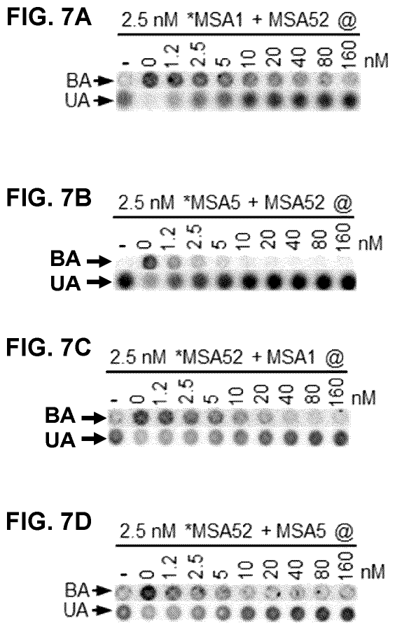

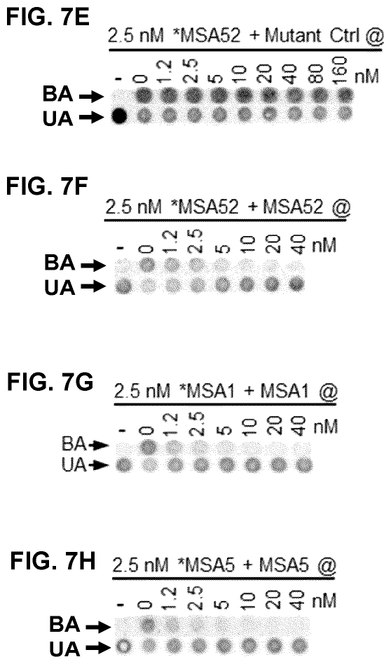

Exploring binding site on spike protein: To better understand the differences in the MSA1, MSA5 and MSA52 binding properties, competition assays were conducted to examine whether the binding sites on the S1 protein of SARS-CoV-2 overlapped for all three aptamers. In this experiment, one aptamer (the aptamer that was being competed against) was radioactively labeled (labeled with * in FIG. 6) and used at 2.5 nM, while the concentration of the other aptamer (the aptamer that was competing) was varied between 0-160 nM. The S1 protein was used at 5 nM. Representative dot blot assays from these competitions are shown in FIG. 7.

Two controls were done to validate the competition: an inactive aptamer control (Mutant Ctrl, FIG. 6; the sequences of all the DNA molecules used in this study are provided in Table 3) that should not compete with any aptamer; and self-competition between radioactive and nonradioactive versions of the same aptamer (*MSA1+MSA1, *MSA5+MSA5, *MSA52+MSA52). The inactive aptamer was indeed unable to compete with MSA52, even at high concentrations (FIG. 6; FIG. 7). In contrast, each radioactive aptamer can be successfully competed out (FIG. 7) by the non-radioactive aptamer in each self-competition, with EC₅₀ values varying between 1-2 nM (FIG. 6).

Based on these control experiments and the competitions between MSA1 and MSA52 or between MSA5 and MSA52, it can be concluded that: (1) MSA5 competes very well with MSA52 (EC₅₀=1.2 and 1.4, respectively for *MSA5+MSA52 and *MSA52+MSA5); (2) MSA1 and MSA52 do not compete well with each other (EC₅₀=12.6 and 9.8, respectively for *MSA1+MSA52 and *MSA52+MSA1). Taking the data presented in FIG. 2 into consideration, the competition results can be interpreted to infer that the binding sites of MSA52 and MSA1 do not significantly overlap while those of MSA52 and MSA5 show significant overlap, though it seems that MSA52 binds to amino acids of the S1 protein that have not been mutated in the current variants of concern that were tested.

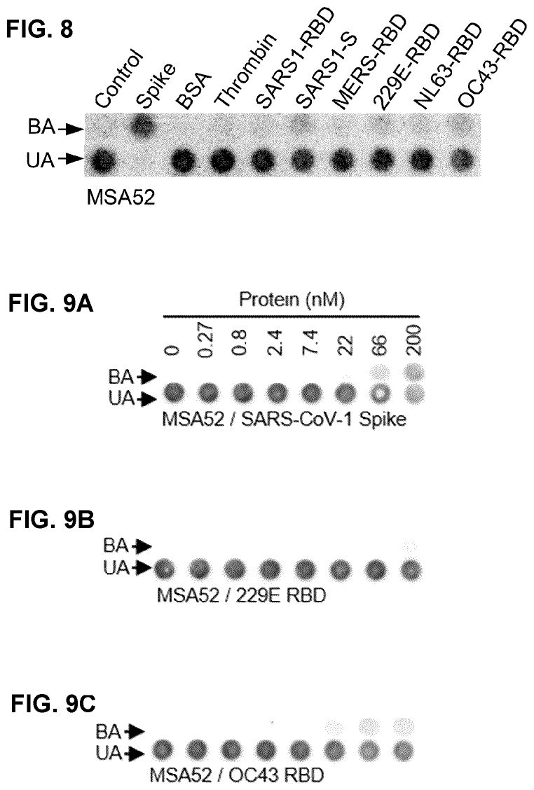

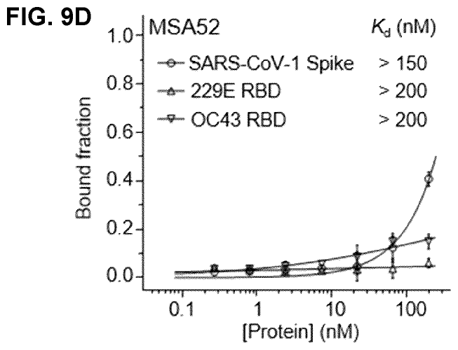

Assessment of binding specificity of MSA52: The specificity of MSA52 was then examined by evaluating its binding to bovine serum albumin (BSA), human-α-thrombin, the RBD and spike (S) proteins of SARS-CoV1, and the RBD proteins of MERS and three seasonal coronavirus 229E, NL63, OC43; the data are provided in FIG. 8. MSA52 exhibits no binding to BSA and thrombin, SARS1-RBD, MERS-RBD or NL63-RBD, but shows very weak binding to the spike protein of SARS-CoV-1, 229E-RBD and OC43-RBD (FIG. 8). However, the binding affinity of MSA52 for these targets was very poor, with K_{d} values greater than 150 nM for the spike protein of SARS-CoV-1 and greater than 200 nM for 229E-RBD and OC43-RBD (FIG. 9). Given that SARS-CoV-1 is no longer in circulation and that the aptamer does not efficiently recognize 229E and OC43, it is clear that MSA52 shows sufficient selectivity for SARS-CoV-2.

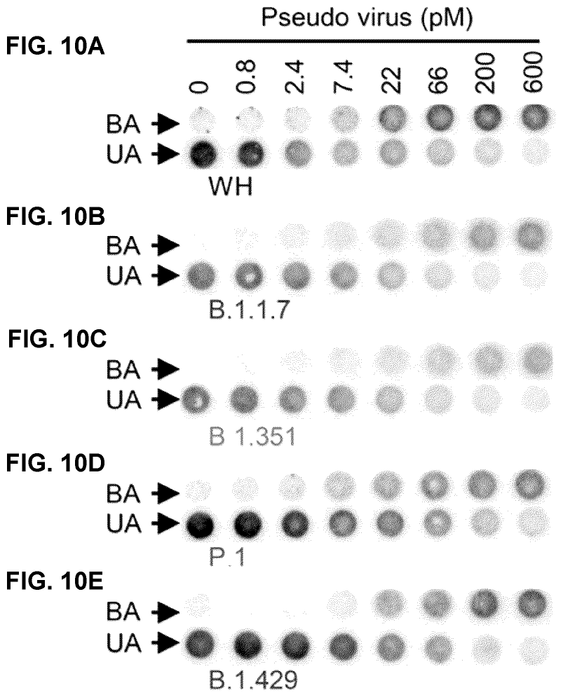

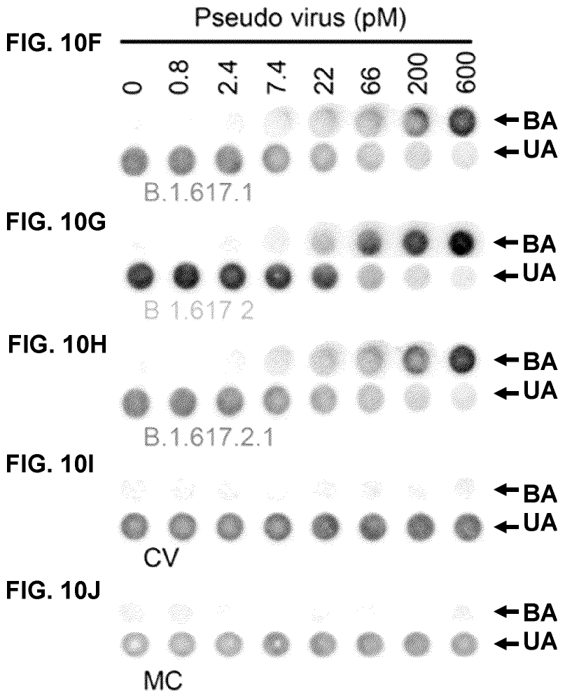

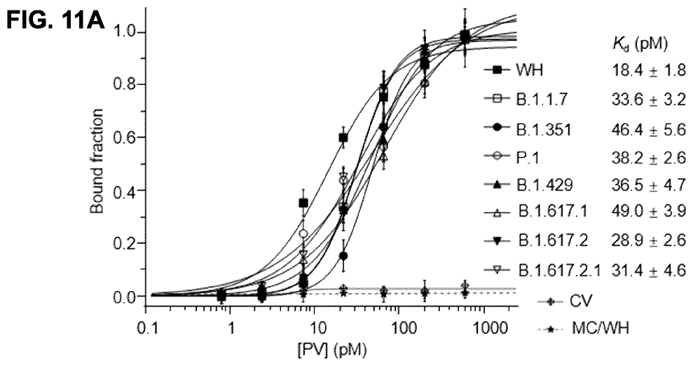

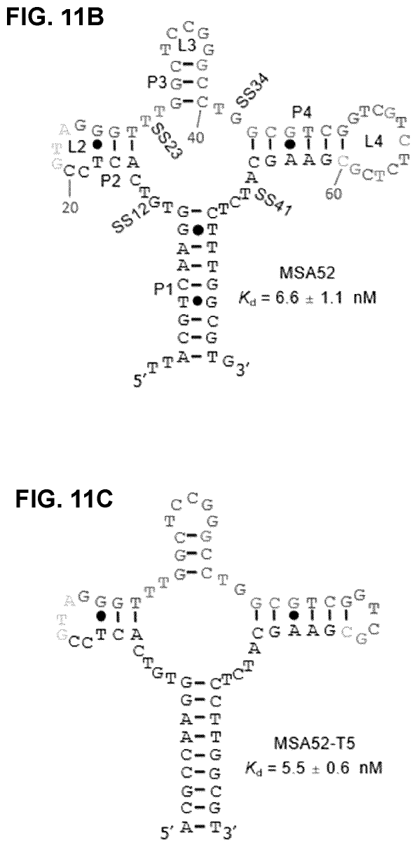

Binding affinity of MSA52 for pseudotyped lentiviruses of SARS-CoV-2 and variants: Next, MSA52 was tested for binding to several pseudotyped SARS-CoV-2 lentiviruses (PLs) that were engineered to display the full trimeric S-proteins of SARS-CoV-2 variants within the viral envelope. These pseudotyped viruses mimic SARS-CoV-2, but they cannot replicate themselves in human cells, allowing them to be handled in biosafety-level-2 labs. The dot blot assays were performed with eight different PLs containing the spike proteins of the original SARS-CoV-2 as well as the B.1.1.7, B.1.351, P.1, B.1,429, B.1.617.1, B.1.617.2, and B.1.617.2.1 (Delta Plus) variants (FIG. 10). The PL of B.1.1.529 (Omicron) variant was not tested as currently it is not available. The same lentivirus that lacks the S-protein was used as a control virus (CV) for this experiment. MSA52 was found to recognize all these PVs with similar affinity but not the CV (FIG. 11A). Excellent K_{d} values were observed for all eight PLs, which ranged from 18.4 pM (WH-PL) to 49.0 pM (B.1.617.1-PL). The increased affinity in comparison to the purified spike proteins can be explained by the fact that each viral particle carries many copies of the S-protein. The copy number of the spike protein on the surface of the SARS-CoV-2 viruses has been reported to be ˜30;[26,27] however, the copy number on the viral particles of the PLs used in this experiment has not been reported. If it is assumed that each PL virus carries 100 copies of the S-protein, the protein-equivalent K_{d} values are estimated to be in the range of 1.84 nM and 4.9 nM, which are close to the K_{d} values for the spike proteins (FIG. 5). More Importantly, MSA52 can recognize fully functional spike proteins of the original SARS-CoV-2 as well as multiple VoCs, even though this aptamer was selected using the purified spike protein from the original virus.

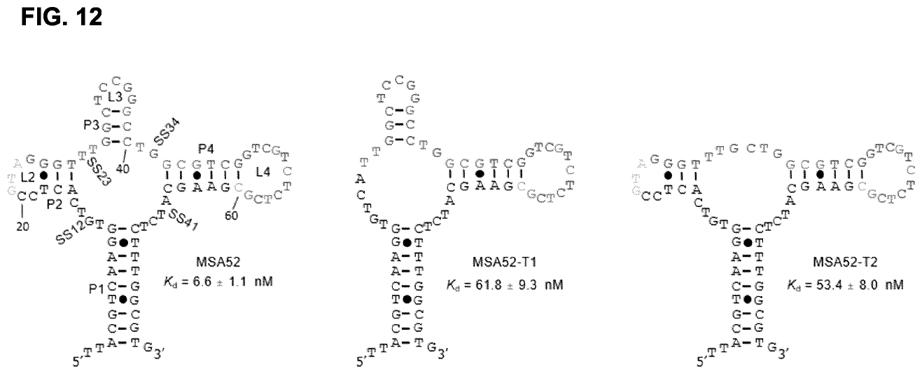

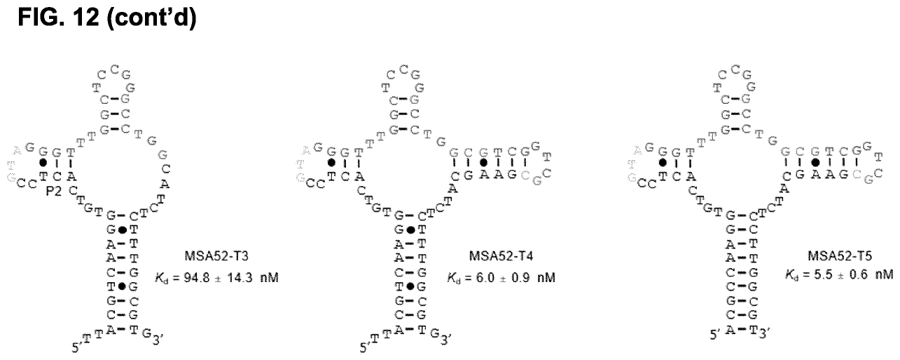

Structure analysis of MSA52 and truncations: The secondary structure of MSA52 was then predicted (FIG. 11B). Overall, the structure contains 4 pairing elements (9-bp P1, 3-bp P2, 3-bp P3 and 6-bp P4) and 5 unpaired elements (4-nt SS12, 2-nt SS23, 2-nt SS34, 4-nt SS41, 6-nt L2, 5-nt L3, 11-nt L4). Five truncation mutants (FIG. 12) were examined by the dot blot assay (named MSA5-T1 to MSA5-T5, respectively) for the binding activity to the S1 protein of the original SARS-CoV-2 virus. The K_{d} value of the full-length MSA52 was first determined to be 6.6 nM. Removing P2-L2 (MSA52-T1; loss of 10 nucleotides; K_{d} of 61.8 nM), P3-L3 (MSA52-T2; loss of 9 nucleotides; K_{d} of 53.4 nM), and P4-L4 (MSA52-T3; loss of 21 nucleotides; K_{d} of 94.8 nM) led to mutants whose binding activities were significantly reduced. However, L4 can be reduced to 4 nucleotides without activity reduction (MSA52-T4; loss of 7 nucleotides; K_{d} of 6.0 nM). Finally, the three unpaired nucleotides next to P1 can be removed and the T⋅G and G⋅T Wobble pairs can be converted to C-G and G-C Watson-Crick pairs without any loss of activity (MSA52-T5, FIG. 11C; loss of 3 nucleotides and change of 2 nucleotides; K_{d} of 5.5 nM). The results above support the proposed four-way junction structure of MSA52.

Low-level random mutations typically occur during the PCR step of the selection process. However, nucleotides whose bases play important roles in the structure and/or binding function of the aptamer show fewer mutations than structurally and/or functionally unimportant bases. The nucleotides in the original random-sequence domain were classified after performing sequence alignment of the top 100 members of the MSA52 family (FIG. 11B): (i) absolutely conserved nucleotides (0 mutations observed): 24-26, 30-32, 37, 40; (ii) highly conserved nucleotides (1 mutation only): 28, 29, 38, 41, 43-45; (iii) somewhat conserved nucleotides (2-3 mutations): 27, 33-36, 39, 42, 46-51, 53, 55-56, 58; (iv) less conserved nucleotides (4-7 mutations): 52, 54, 57, 59; (v) least conserved nucleotides (9-29 mutations): 21-23, 60. The conservation pattern seen with the sequence of MSA52 is consistent with the truncation data: any truncation mutant that loses the highly conserved nucleotides experienced significantly reduced binding affinity. It can be further reasonably considered that the nucleotides that directly engage the spike protein for molecular recognition are located within the 22-nt segment starting with the 24^{th} G residue within P2-L2 and ending with the 45^{th} G residue within P4 (FIG. 11). More detailed structural and functional analysis of these nucleotides constitutes a future research interest via the examination of more mutated sequence constructs.

Comparison of binding affinity of MSA52 and 10 published aptamers for spike proteins of selective variants of SARS-CoV-2: To demonstrate the uniqueness of MSA52 in recognition specificity, a comparison study was conducted in which the binding affinity of 10 published spike-binding DNA aptamers was determined for the spike proteins of the original SARS-CoV-2 as well as the B.1.1.7, B.1.351, P.1, B.1.617.2 and the latest B.1.1.529 (Omicron) variants using dot blot assays (Table 4). Five of these aptamers were from inventors and the other five were randomly selected from 5 other aptamer studies. MSA52 was found to recognize all these spike proteins with similar affinity, with K_{d} ranging from 3.6 to 10.2 nM. In contrast, no other aptamers exhibited universally excellent affinity for all five variants. For example, MSA1 showed excellent affinity for B.1.1.7S (1.2 nM) and B.1.617.2S (3.2 nM) but reduced affinity for WHS (19.8 nM) and poor affinity for P.1S (75.2 nM) and B.1.351S (>200 nM). The second-best aptamer in terms of universal affinity is MSA3, exhibiting K_{d} values ranging from 2.0 to 36.4 nM. In Table 4, the top 3 aptamers for each variant are also indicated. MSA52 ranks first for P.1S, B.1.617.2S and B.1.1.529S; a close second for two variants: B.1.351S (8.5 nM for MSA52 vs. 8.2 nM for MSA5) and WHS (3.6 nM for MSA52 vs. 3.3 nM for SP6); and a reasonably close third for B.1.1.7S (3.8 nM for MSA52 vs. 1.2 nM for MSA1 and 2.0 nM for MSA3). This head-to-head comparison study further demonstrates two important traits of MSA52: (1) its affinity is among the best of the aptamers for spike proteins, and (2) it is highly unique in terms of exhibiting uniformly excellent affinity for the five variants of the spike protein.

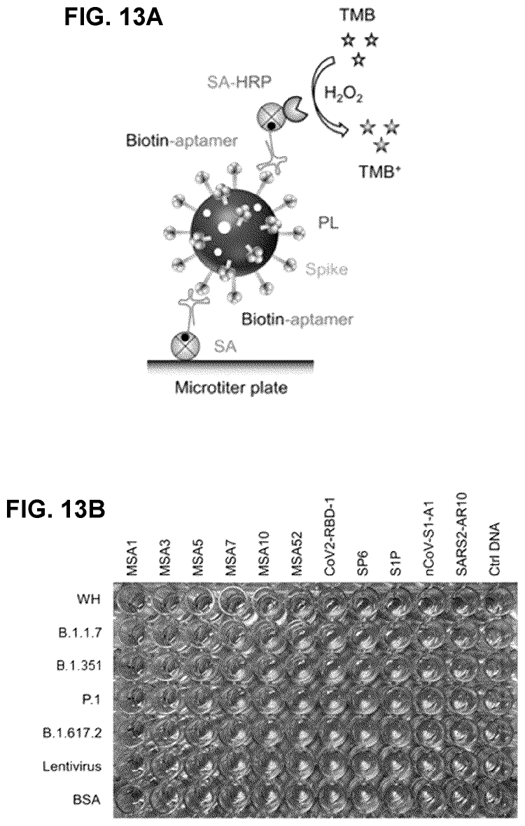

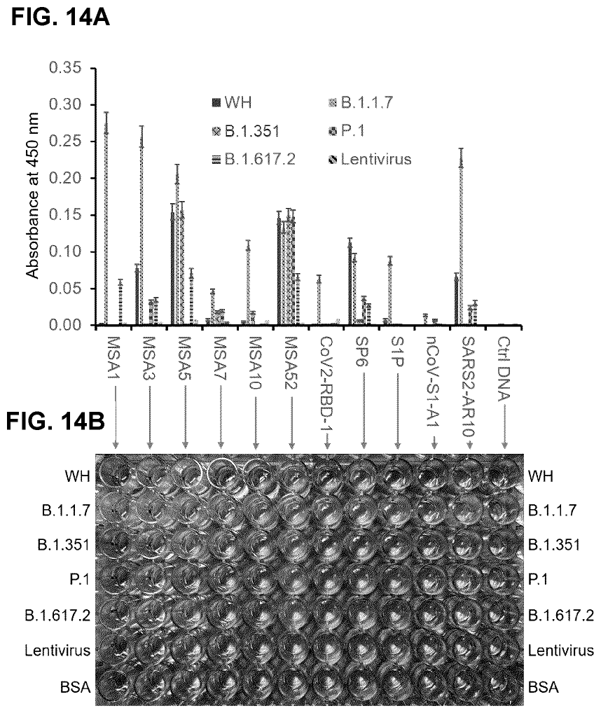

Comparison of analytical performance of MSA52 and 10 published aptamers in sandwich assays for detection of pseudotyped lentiviruses of SARS-CoV-2 variants: Another comparison study was conducted to demonstrate the analytical utility of MSA52 for the detection of pseudotyped lentiviruses of the original SARS-CoV-2 (WH) and the B.1.1.7, B.1.351, P.1 and B.1.617.2 variants. Because each viral particle carries multiple spike proteins on its surface, inventors designed a sandwich assay that uses two identical biotinylated aptamers to bind a single viral particle (FIG. 13). Each of the 11 aptamers in Table 4 was biotinylated at the 5′ end and immobilized onto a 96-well microtiter plate coated with streptavidin. The second biotinylated aptamer was tagged with horseradish peroxidase (HRP) conjugated to streptavidin. The presence of viral particles led to immobilization of HRP onto the plate, which oxidized 3,3′,5,5′-tetramethylbenzidine (TMB) in the presence of H₂O₂. Three controls were included in this experiment: BSA as a general protein target, lentivirus that does not express any spike protein and polyA25 as a DNA control. After quenching the reaction with H₂SO₄, the blue-colored oxidized TMB turned yellow and was measured at 450 nm. FIG. 14A plots the absorbance at 450 nm (A₄₅₀) in binding reaction mixtures containing a specific aptamer and a PL (2 pM). The A₄₅₀ value of each reaction mixture was corrected for background by subtracting A₄₅₀ observed for the matching BSA reaction mixture. FIG. 14B shows a representative image of the 96-well microtiter plate following the quenching reaction with H₂SO₄ (the image was processed with Photoshop for enhanced visual effect; the original image is provided in FIG. 13B).

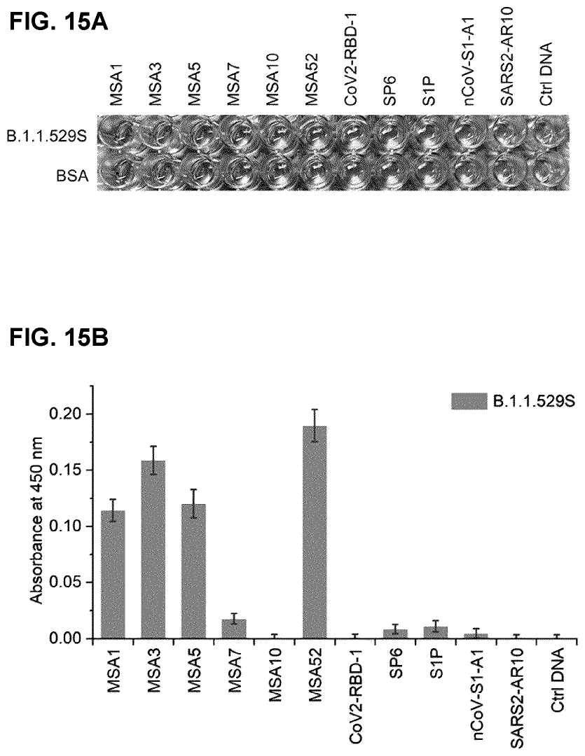

The data in FIG. 14A clearly shows that only MSA52 was able to produce a high signal (A₄₅₀ greater than 0.05) for all five variants, and nearly all aptamers were able to detect the B.1.1.7 variant under the test conditions. The next best aptamer was MSA5, which was able to detect 4 out of 5 variants but failed to detect the P.1 variant. In fact, except for MSA52, nearly all the other aptamers either produced a very weak signal (MSA1, MSA5, MSA10, CoV2-RBD-1, SIP, and nCoV-S1-A1) or a significantly smaller signal (MSA3, MSA7, SP6 and SARS2-AR10) with the P.1 variant. B.1.351 was another variant that most of the aptamers had difficulty in recognizing: only MSA5 and MSA52 produced a high-level signal while the others produced very weak signals. Another interesting observation is that all the aptamers with a K_{d} of ≤11.5 nM in Table 4 for a variant could produce a high-level signal. MSA52 meets this criterion for all five variants, MSA5 meets this criterion for 4 variants, while the remaining aptamers only satisfy this criterion for 0 (MSA10 and nCoV-S1-A1), 1 (MSA7, CoV2-RBD-1 and SIP) and 2 (MSA1, MSA3, SP6 and SARS2-AR10) variants. Inventors also tested this sandwich assay for B.1.1.529 (Omicron) variant using trimeric spike protein because the pseudotyped viruses for omicron variant is not currently available. MSA52 was the best among the four aptamers (MSA52, MSA1, MSA3, and MSA5) that showed high-level signals (FIGS. 15A and 15B). In contrast, the other aptamer either had very weak signals or no signal. These observations are consistent with the dot blot results in the Table 4 (MSA52 ranked first for Omicron).

In summary, parallel selection was performed using an aptamer pool from a previous SELEX experiment with the wild-type S1 protein of SARS-CoV-2, followed by high-throughput DNA sequencing and bioinformatic analysis, in search for DNA aptamers that universally (i.e., non-discriminatively) recognize spike proteins of SARS-CoV-2 variants. This effort has led to the discovery of a unique DNA aptamer, named MSA52, that can bind spike proteins of the wildtype SARS-CoV-2 and its eight current variants of concern, including B.1.1.7, B.1.351, P.1, B.1.429, B.1.617.1 B.1.617.2, B.1.617.2.1 and B.1.1.529, while still retaining selectivity against non-SARS-CoV-2 spike proteins. This aptamer was discovered using a rapid one-round reselection against four variants of the SARS-CoV-2 spike protein, but was observed to show high affinity binding to both these four variants as well as three other variants (including the latest Omicron variant) that were not present during reselection, indicating universally high affinity for both known and emerging VoCs. The entire discovery process (SELEX, sequencing and bioinformatic analysis) was very rapid, taking less than a week. Competition assays showed that this unique aptamer should bind to key amino acids present in all variants, allowing universal recognition of the spike protein of the current and emerging variants of concern, making it well-suited as a molecular recognition element for developing diagnostic and therapeutic solutions to this constantly evolving coronavirus.

While the present disclosure has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present disclosure is found to be defined differently in a document incorporated herein by reference, the definition provided herein is to serve as the definition for the term.

### TABLES

TABLE 1A
DNA sequences in the combined pools of members of
the MSA52 aptamer family selected for WHS, UKS,
BZS, SAS, and CAS ranked by their frequency.

| SEQ | |
|---|---|
| ID | |
| No | Sequence^{[a]} |
| 1 | GTAGGGTTTGGCTCCGGGCCTGGCGTCGGTCGTCTCTCGC |
| 2 | -TAGGGTTTGGCTCCGGGCCTGGCGTCGGTCGTCTCTCGC |
| 3 | ATAGGGTTTGGCTCCGGGCCTGGCGTCGGTCGTCTCTCGC |
| 4 | GTAGGGTTTGGCTCCGGGCCTGGCGTCGGTCGTCTCTCGT |
| 5 | TTAGGGTTTGGCTCCGGGCCTGGCGTCGGTCGTCTCTCGC |
| 6 | GTAGGGTTTGGCTCCGGGCCTGGCGTCGGTCGTCTTTCGC |
| 7 | GTAGGGTTTGGCTCCGGGCCTGGCGTCGGTCGTCTCTTGC |
| 8 | G-CGGGTTTGGCTCCGGGCCTGGCGTCGGTCGTCTCTCGC |
| 9 | GTAGGGTTTGGCTCCGGGCCTGGCGTCGGTCGTCTCCCGC |
| 10 | GCAGGGTTTGGCTCCGGGCCTGGCGTCGGTCGTCTCTCGC |
| 11 | CTAGGGTTTGGCTCCGGGCCTGGCGTCGGTCGTCTCTCGC |
| 12 | GTAGGGTTTGGCTCCGGGCCTGGCGTCGGTTGTCTCTCGC |
| 13 | GTAGGGTTTGGCTCCGGGCCTGGCGTCGGTCATCTCTCGC |
| 14 | GTAGGGTTTGGCTCCGGGCCTGGCGTCGGTCGTCCCTCGC |
| 15 | GTGGGGTTTGGCTCCGGGCCTGGCGTCGGTCGTCTCTCGC |
| 16 | GTAGGGTTTGGCTCCGGGCCTGGCGTAGGTCGTCTCTCGC |
| 17 | GAAGGGTTTGGCTCCGGGCCTGGCGTCGGTCGTCTCTCGC |
| 18 | GTAGGGTTTGGCTCCGGGCCTGGCGTCGGTCGCCTCTCGC |
| 19 | GTAGGGTTTGGCTCCGGGCCTGGCGTCGGTCGTCTCTCGA |
| 20 | AC-GGGTTTGGCTCCGGGCCTGGCGTCGGTCGTCTCTCGC |
| 21 | GTAGGGTTTGGCTCCGGGCCTGGCGTCGGTCGTCTCTCTC |
| 22 | GTAGGGTTTGGCTCCGGGCCTGGCGTCGGTCGTTTCTCGC |
| 23 | ATAGGGTTTGGCTCCGGGCCTGGCGTCGGTCGTCTCTCGT |
| 24 | -T-GGGTTTGGCTCCGGGCCTGGCGTCGGTCGTCTCTCGC |
| 25 | GTAGGGTTTGGCTCAGGGCCTGGCGTCGGTCGTCTCTCGC |
| 26 | GTAGGGTTTGGCTCCGGGCCTGGCGTCGGTCGTCTC-CTC |
| 27 | GTAGGGTTTGGCTCCGGGCCTGGCGTCGGTCCTCTCTCGC |
| 28 | GTAGGGTTTGGCTCCGGGCCTGGCGACGGTCGTCTCTCGC |
| 29 | GTAGGGTTTGGCTCCGGGCCTGGCGTCGGTCTTCTCTCGC |
| 30 | -CAGGGTTTGGCTCCGGGCCTGGCGTCGGTCGTCTCTCGC |
| 31 | --AGGGTTTGGCTCCGGGCCTGGCGTCGGTCGTCTCTCGC |
| 32 | GTAGGGTTTGGCTCCGGACCTGGCGTCGGTCGTCTCTCGC |
| 33 | GTAGGGTTTGGCTCCGGGCCT-GCGTCGGTCGTCTCTCGC |
| 34 | GTAGGGTTTGGCTCCGGGCCTGGCGTCGGTCGTCTCTCAC |
| 35 | GTAGGGTTTGGCTCCGGGCCTGGCGTCGGTCGTCTCTCGG |
| 36 | ACTGGGTTTGGCTCCGGGCCTGGCGTCGGTCGTCTCTCGC |
| 37 | GTAGGGTTTGGCTCCGGGCCTGGCGTCGGTCGTCTCTAGC |
| 38 | GTAGGGCTTGGCTCCGGGCCTGGCGTCGGTCGTCTCTCGC |
| 39 | GTAGGGTTTGGCTCCGGGCCTGGCGTCGGTCGTCTCTCCC |
| 40 | ATGGGGTTTGGCTCCGGGCCTGGCGTCGGTCGTCTCTCGC |
| 41 | GTAGGGTTTGGCTCCGGGCCTGGCGTCGTTCGTCTCTCGC |
| 42 | GTAGGGTTTGGCTCCGGGCCTAGCGTCGGTCGTCTCTCGC |
| 43 | GTAGGGTTTGGCTCCGGGCCTGGCGTCGGTAGTCTCTCGC |
| 44 | GGAGGGTTTGGCATCGGGCCTGGCGTCGGTCGTCTCTCGC |
| 45 | GTAGGGTTTGGCTCCGGGCCTGGCTTCGGTCGTCTCTCGC |
| s46 | GTAGGGTTTGGCTCCGGGCCTGGCGCCGGTCGTCTCTCGC |
| 47 | GTAGGGTTTGGCTCCGGGCCCGGCGTCGGTCGTCTCTCGC |
| 48 | -TGGGGTTTGGCTCCGGGCCTGGCGTCGGTCGTCTCTCGC |
| 49 | ACAGGGTTTGGCTCCGGGCCTGGCGTCGGTCGTCTCTCGC |
| 50 | GTAGGGTCTGGCTCCGGGCCTGGCGTCGGTCGTCTCTCGC |
| 51 | GTAGGGTTTGGCTCCGGGCCTGGCGTCGATCGTCTCTCGC |
| 52 | GTAGGGTTTGGCTCCGGGCCTGGCGTCGGTCGTCTCCCGT |
| 53 | ATAGGGTTTGGCTCCGGGCCTGGCGTCGGTCGTTTCTCGC |
| 54 | ATAGGGTTTGGCTCCGGGCCTGGCGTCGGTCTTCTCTCGC |
| 55 | GTAGGGTTTGGCTACGGGCCTGGCGTCGGTCGTCTCTCGC |
| 56 | GTAGGGTTTGGCTCCGGGCCTGGCGTCGGTCGTGTCTCGC |
| 57 | -TAGGGTTTGGCTCCGGGCCTGGCGTCGTTCGTCTCTCGC |
| 58 | -TAGGGTTTGGCTCCGGGCCTGGCGTCGGTCGTCTCTCGT |
| 59 | TCGGGGGGGTTTGGCTCCGGGCCTGGCGTCGGTCGTCTCTCGC |
| 60 | -TAGGGTTTGGCTCCGGGCCTGGCGTCGGTCGTCTTTCGC |
| 61 | GTAGGGTTTGGCTCCGGGCCTGGCGTCGGCCGTCTCTCGC |
| 62 | GTTGGGTTTGGCTCCGGGCCTGGCGTCGGTCGTCTCTCGC |
| 63 | TCCGTAGGGTTTGGCTCCGGGCCTGGCGTCGGTCGTCTCTCGC |
| 64 | GTAGGGTTTGGCCCCGGGCCTGGCGTCGGTCGTCTCTCGC |
| 65 | GTAGGGTTTGGCTCCAGGCCTGGCGTCGGTCGTCTCTCGC |
| 66 | GTAGGGTTCGGCTCCGGGCCTGGCGTCGGTCGTCTCTCGC |
| 67 | GTAGGGTTTGGCTCCGGGCCTGGCGTCGGTCGTCTCTC-- |
| 68 | -TCGGGTTTGGCTCCGGGCCTGGCGTCGGTCGTCTCTCGC |
| 69 | GTAGGGTTTGGCTCCGGGCCTGGCGTCGGACGTCTCTCGC |
| 70 | GTAGGGTTTGGCTCCGGGCCTGGCGTTGGTCGTCTCTCGC |
| 71 | GTAGGGTTTGGCTCCGGGCCTGGCGTCGGTCGTCTATCGC |
| 72 | ATAGGGTTTGGCTCCGGGCCTGGCGTCGGTTGTCTCTCGC |
| 73 | G-CGGGTTTGGCTCCGGGCCTGGCGTCGGTCGTCTCTCGT |
| 74 | GTAGGGTTTGGCTCCGGGCCTGGCGTCAGTCGTCTCTCGC |
| 75 | GTAGGGTTTGGCTTCGGGCCTGGCGTCGGTCGTCTCTCGC |
| 76 | GTAGGGTTTGGCTCCGGGCCTGGCGTCGGTCGTCGCTCGC |
| 77 | GTAGGGTTTGGCTCCGGGCCTGGCGTCGGTCGACTCTCGC |
| 78 | GTAGGGTTTGGCTCCGGGCCTGGCGTCGGTCGTCACTCGC |
| 79 | GTAGGGTTTGGCTCCGGGCCTGGCGTCTGTCGTCTCTCGC |
| 80 | ATCGGGTTTGGCTCCGGGCCTGGCGTCGGTCGTCTCTCGC |
| 81 | GTAGGGTTTGGCTCCGGGCCTGGCGTCGGTCGTCTC-CCC |
| 82 | -TAGGGTTTGGCTCCGGGCCTGGCGTCGGTCGTCTCTTGC |
| 83 | TTAGGGTTTGGCTCCGGGCCTGGCGTCGGTCGTCTCTCGT |
| 84 | GTAGGGTTTGGCTCTGGGCCTGGCGTCGGTCGTCTCTCGC |
| 85 | G--GGGTTTGGCTCCGGGCCTGGCGTCGGTCGTCTCTCGC |
| 86 | AT-GGGTTTGGCTCCGGGCCTGGCGTCGGTCGTCTCTCGC |
| 87 | GTAGGGTTTGGCTCCGGGCCTGACGTCGGTCGTCTCTCGC |
| 88 | -T-GGGATTGGCTCCGGGCCTGGCGTCGGTCGTCTCTCGC |
| 89 | GTAGGGTTTGGCTCCGGGTCTGGCGTCGGTCGTCTCTCGC |
| 90 | ATAGGGTTTGGCTCCGGGCCTGGCGTCGGTCATCTCTCGC |
| 91 | CACTGGGTTTGGCTCCGGGCCTGGCGTCGGTCGTCTCTCGC |
| 92 | GCAGGGTTTGGCTCCGGGCCTGGCGTCGGTCGTCTCTCGT |
| 93 | G--GGGATTGGCTCCGGGCCTGGCGTCGGTCGTCTCTCGC |
| 94 | GTAGGGTTTGGCTCCGGGACTGGCGTCGGTCGTCTCTCGC |
| 95 | GTAGGGTTTGGCTCCGGGCCTGGCGTCGGTCGTCTCCCCC |
| 96 | -TAGGGTTTGGCTCCGGGCCTGGCGTCGGCCGTCTCTCGC |
| 97 | -TAGGGTTTGGCTCCGGGCCTGGCGTCGGTCATCTCTCGC |
| 98 | GTAGGGTTTGGCTCCGGGCCTGGTGTCGGTCGTCTCTCGC |
| 99 | GTAGGGTTTGGCTCCTGGCCTGGCGTCGGTCGTCTCTCGC |
| 100 | GTAGGGATTGGCTCCGGGCCTGGCGTCGGTCGTCTCTCGC |

^{[a]}: Each sequence contains primer regions of TTACGTCAAGGTGTCACTCC (SEQ ID NO: 102) and GAAGCATCTCTTTGGCGTG (SEQ ID NO: 132) at the 5′ end and 3′ end, respectively. Each point mutation in relation to the top ranked sequence is in bold.

TABLE 1B
DNA sequences in the combined pools of members of the MSA52 aptamer family
selected for WHS, UKS, BZS, SAS, and CAS ranked by their frequency.

| SEQ | | | | | | | | | | |
|---|---|---|---|---|---|---|---|---|---|---|
| ID | Rank^{[b]} | | | | | Frequency (10⁻⁶) | | | | |
| No | WHS | UKS | BZS | SAS | CAS | WHS | UKS | BZS | SAS | CAS |
| 1 | 46 | 24 | 17 | 19 | 14 | 1382 | 3203 | 4447 | 4294 | 3860 |
| 2 | 455 | 295 | 201 | 273 | 155 | 95 | 171 | 280 | 206 | 293 |
| 3 | 460 | 254 | 176 | 207 | 128 | 94 | 196 | 323 | 293 | 336 |
| 4 | 757 | 354 | 297 | 332 | 256 | 54 | 139 | 196 | 161 | 164 |
| 5 | 1128 | 662 | 468 | 599 | 475 | 34 | 60 | 109 | 83 | 74 |
| 6 | 1756 | 697 | 780 | 775 | 553 | 17 | 55 | 59 | 61 | 57 |
| 7 | 1851 | 1313 | 1077 | 1484 | 874 | 16 | 23 | 39 | 25 | 29 |
| 8 | 2021 | 1070 | 631 | 612 | 492 | 13 | 31 | 77 | 81 | 69 |
| 9 | 2041 | 1253 | 1238 | 1383 | 1095 | 13 | 24 | 32 | 28 | 21 |
| 10 | 2172 | 1000 | 620 | 957 | 729 | 12 | 34 | 79 | 46 | 38 |
| 11 | 2203 | 2663 | 1296 | 1595 | 961 | 12 | 9 | 31 | 23 | 24 |
| 12 | 2785 | 1168 | 1700 | 1800 | 1331 | 8 | 27 | 22 | 19 | 16 |
| 13 | 2788 | 1471 | 1797 | 1602 | 983 | 8 | 20 | 20 | 23 | 24 |
| 14 | 2790 | 1252 | 1200 | 1483 | 1040 | 8 | 24 | 34 | 25 | 22 |
| 15 | 3110 | 2141 | 1917 | 2129 | 1042 | 7 | 12 | 18 | 16 | 22 |
| 16 | 3132 | 2134 | 1698 | 2818 | 1228 | 7 | 12 | 22 | 11 | 17 |
| 17 | 3140 | 1763 | 2434 | 1791 | 1150 | 7 | 15 | 13 | 19 | 19 |
| 18 | 3456 | 1663 | 1699 | 1728 | 2087 | 6 | 17 | 22 | 20 | 9 |
| 19 | 3540 | 2135 | 2044 | 4571 | 1230 | 6 | 12 | 16 | 6 | 17 |
| 20 | 4025 | 1238 | 1406 | 1518 | 854 | 5 | 24 | 27 | 24 | 29 |
| 21 | 4054 | 1472 | 1201 | 2000 | 637 | 5 | 20 | 34 | 17 | 47 |
| 22 | 4087 | 3157 | 3240 | 3431 | 1618 | 5 | 8 | 9 | 8 | 12 |
| 23 | 4096 | 20213 | 7714 | 5234 | 1972 | 5 | <1 | 4 | 5 | 9 |
| 24 | 4101 | 2464 | 1731 | 4761 | 1182 | 5 | 11 | 22 | 6 | 19 |
| 25 | 4844 | 8748 | 2042 | 26086 | 2082 | 5 | 3 | 16 | <1 | 9 |
| 26 | 4917 | 3156 | 2786 | 2427 | 1616 | 4 | 8 | 11 | 13 | 12 |
| 27 | 5000 | 2692 | 1598 | 4569 | 1039 | 4 | 9 | 23 | 6 | 22 |
| 28 | 5026 | 2133 | 2225 | 5576 | 2083 | 4 | 12 | 14 | 5 | 9 |
| 29 | 5102 | 5173 | 2463 | 3432 | 1041 | 4 | 5 | 13 | 8 | 22 |
| 30 | 5144 | 21204 | 3766 | 24575 | 4702 | 4 | <1 | 7 | <1 | 3 |
| 31 | 6321 | N | 17877 | N | N | 4 | N | <1 | N | N |
| 32 | 6417 | 22888 | N | 7532 | 11522 | 4 | <1 | N | 4 | <1 |
| 33 | 6418 | N | N | N | N | 4 | N | N | N | N |
| 34 | 6548 | 1664 | 1599 | 1999 | 1229 | 2 | 17 | 23 | 17 | 17 |
| 35 | 6569 | 1777 | 3239 | 5580 | 2526 | 2 | 15 | 9 | 5 | 7 |
| 36 | 6616 | 2559 | 1647 | 3268 | 9562 | 2 | 9 | 22 | 8 | <1 |
| 37 | 6617 | 3867 | 3238 | 5579 | 1096 | 2 | 6 | 9 | 5 | 21 |
| 38 | 6623 | 8745 | 3235 | 2426 | 3269 | 2 | 3 | 9 | 13 | 5 |
| 39 | 6671 | 2693 | 5228 | 4570 | 1617 | 2 | 9 | 5 | 6 | 12 |
| 40 | 6737 | 8069 | 18301 | 3704 | 1988 | 2 | 3 | <1 | 7 | 9 |
| 41 | 6764 | 2136 | 2045 | 3433 | 834 | 2 | 12 | 16 | 8 | 31 |
| 42 | 6794 | 22892 | 8593 | 26089 | N | 2 | <1 | 4 | <1 | N |
| 43 | 6823 | 3865 | 3944 | 12354 | 2086 | 2 | 6 | 7 | 2 | 9 |
| 44 | 6825 | N | 20464 | 7463 | 3224 | 2 | N | <1 | 4 | 5 |
| 45 | 6826 | 2695 | 3949 | 26100 | 2091 | 2 | 9 | 7 | <1 | 9 |
| 46 | 6832 | 22896 | 8594 | 4568 | 2084 | 2 | <1 | 4 | 6 | 9 |
| 47 | 6843 | 22891 | 20717 | 12352 | 5032 | 2 | <1 | <1 | 2 | 3 |
| 48 | 6861 | 24824 | 5568 | 7994 | 5381 | 2 | <1 | 5 | 4 | 3 |
| 49 | 7049 | 7672 | 3542 | 4195 | 1689 | 2 | 3 | 7 | 6 | 10 |
| 50 | 8000 | 22882 | 8591 | 5574 | 11515 | 2 | <1 | 4 | 5 | <1 |
| 51 | 8110 | 22898 | 20721 | 26091 | 2525 | 2 | <1 | <1 | <1 | 7 |
| 52 | 8111 | N | N | N | N | 2 | N | N | N | N |
| 53 | 9039 | N | N | N | N | 2 | N | N | N | N |
| 54 | 9040 | N | 18072 | N | 4556 | 2 | N | <1 | N | 3 |
| 55 | 9627 | 5170 | 2462 | 26085 | 3270 | 2 | 5 | 13 | <1 | 5 |
| 56 | 9628 | 22904 | 20727 | N | 11529 | 2 | <1 | <1 | N | <1 |
| 57 | 9730 | N | 21117 | N | 11857 | 2 | N | <1 | N | <1 |
| 58 | 10472 | 5236 | 5295 | 2839 | 988 | <1 | 5 | 5 | 11 | 24 |
| 59 | 10474 | 23821 | 4036 | 3124 | 1245 | <1 | <1 | 7 | 10 | 17 |
| 60 | 10476 | 23260 | 8716 | 4607 | 2558 | <1 | <1 | 4 | 6 | 7 |
| 61 | 10477 | 1927 | 2785 | 2591 | 1461 | <1 | 14 | 11 | 12 | 14 |
| 62 | 10491 | 8790 | 3250 | 2598 | 5060 | <1 | 3 | 9 | 12 | 3 |
| 63 | 10536 | 5305 | 8812 | 7716 | 12173 | <1 | 5 | 4 | 4 | <1 |
| 64 | 10556 | 8747 | 3237 | 3091 | 5029 | <1 | 3 | 9 | 10 | 3 |
| 65 | 10573 | 22887 | 20715 | 26087 | 5031 | <1 | <1 | <1 | <1 | 3 |
| 66 | 10599 | 22883 | 2223 | 3090 | 5027 | <1 | <1 | 14 | 10 | 3 |
| 67 | 10665 | 2385 | 1915 | 3857 | 934 | <1 | 11 | 18 | 7 | 26 |
| 68 | 10669 | N | 8943 | 5744 | 12433 | <1 | N | 4 | 5 | <1 |
| 69 | 10678 | 3155 | 3943 | 2819 | 5034 | <1 | 8 | 7 | 11 | 3 |
| 70 | 10729 | 2694 | 2046 | 2428 | 2090 | <1 | 9 | 16 | 13 | 9 |
| 71 | 10785 | 3866 | 8595 | 5578 | 1163 | <1 | 6 | 4 | 5 | 19 |
| 72 | 10794 | 20214 | 7717 | 6917 | 9824 | <1 | <1 | 4 | 4 | <1 |
| 73 | 10801 | 22512 | 3200 | 25741 | 4919 | <1 | <1 | 9 | <1 | 3 |
| 74 | 10895 | 22897 | 5225 | 5577 | 3272 | <1 | <1 | 5 | 5 | 5 |
| 75 | 10897 | 22910 | 3243 | 7536 | 11532 | <1 | <1 | 9 | 4 | <1 |
| 76 | 10915 | 5172 | 20723 | 7534 | N | <1 | 5 | <1 | 4 | N |
| 77 | 10949 | 5171 | 20722 | 7533 | 11525 | <1 | 5 | <1 | 4 | <1 |
| 78 | 10950 | 8751 | 5226 | 3430 | 2088 | <1 | 3 | 5 | 8 | 9 |
| 79 | 10951 | 3158 | 2787 | 3092 | 1164 | <1 | 8 | 11 | 10 | 19 |
| 80 | 10998 | 20292 | 4820 | 23795 | 9899 | <1 | <1 | 5 | <1 | <1 |
| 81 | 11026 | N | 8596 | 12356 | 11527 | <1 | N | 4 | 2 | <1 |
| 82 | 11030 | 3921 | 21116 | 26470 | 11856 | <1 | 6 | <1 | <1 | <1 |
| 83 | 11041 | 26005 | 9590 | 13599 | 3557 | <1 | <1 | 4 | 2 | 5 |
| 84 | 11050 | 8757 | 3242 | 3859 | 3275 | <1 | 3 | 9 | 7 | 5 |
| 85 | 11119 | 22782 | 8545 | 4552 | 11431 | <1 | <1 | 4 | 6 | <1 |
| 86 | 11158 | 20431 | 18316 | 11470 | 9984 | <1 | <1 | <1 | 2 | <1 |
| 87 | 11172 | N | 20718 | 12353 | 11523 | <1 | N | <1 | 2 | <1 |
| 88 | 11177 | 24782 | N | 7980 | 13081 | <1 | <1 | N | 4 | <1 |
| 89 | 11192 | 2137 | 8597 | 5585 | 11531 | <1 | 12 | 4 | 5 | <1 |
| 90 | 11232 | 20211 | 4793 | 11387 | 2333 | <1 | <1 | 5 | 2 | 7 |
| 91 | 11250 | 20945 | 3753 | 11636 | 10316 | <1 | <1 | 7 | 2 | <1 |
| 92 | 11274 | 22276 | 5118 | 12095 | 11049 | <1 | <1 | 5 | 2 | <1 |
| 93 | 11295 | 8671 | N | 25920 | 3235 | <1 | 3 | N | <1 | 5 |
| 94 | 11312 | 22889 | N | 26088 | N | <1 | <1 | N | <1 | N |
| 95 | 11314 | 22901 | N | N | N | <1 | <1 | N | N | N |
| 96 | 11323 | 23256 | N | N | N | <1 | <1 | N | N | N |
| 97 | 11324 | 23257 | N | 26468 | 5114 | <1 | <1 | N | <1 | 3 |
| 98 | 11385 | 8755 | 20732 | 5584 | 11530 | <1 | 3 | <1 | 5 | <1 |
| 99 | 11397 | 5175 | 1916 | 7535 | 3274 | <1 | 5 | 18 | 4 | 5 |
| 100 | 11465 | 22881 | 20709 | 7531 | N | <1 | <1 | <1 | 4 | N |

^{[b]}N refers to “not detected”.

TABLE 2
Ratio paired t-test results comparing the top 15 MSA52 cluster
members in WHS, UKS, BZS, SAS and CAS pools as compared
to the Round 13 pool. MSA52 cluster member frequencies
are not significantly different between the Round 13 reference
population and the WHS population. UKS, BZS, SAS and CAS
populations of MSA52 are significantly enriched as compared
to the Round 13 reference population.

| | WHS | UKS | BZS | SAS | CAS |
|---|---|---|---|---|---|
| P value | 0.7718 | <0.0001 | <0.0001 | <0.0001 | <0.0001 |
| Geometric | 1.053 | 2.209 | 3.435 | 2.877 | 2.847 |
| Mean of Ratios | | | | | |
| SD of log(ratio) | 0.2912 | 0.2467 | 0.3188 | 0.3005 | 0.2961 |
| 95% CI | 0.7260 to | 1.613 to | 2.288 to | 1.961 to | 1.951 to |
| | 1.526 | 3.026 | 5.159 | 4.220 | 4.153 |

TABLE 3
All the synthetic oligonucleotides used in this study.

| SEQ ID | | | |
|---|---|---|---|
| NO | Selection | | |
| 101 | DNA library (79 nt) | | TTACGTCAAG GTGTCACTCC-N₄₀- |
| | | | GAAGCATCTC TTTGGCGTG |
| 102 | Forward primer FP1 (20 nt) | | TTACGTCAAG GTGTCACTCC |
| 103 | Forward primer FP2 (20 nt) | | FAM-TTACGTCAAG GTGTCACTCC |
| 104 | Reverse primer RP1 (19 nt) | | CACGCCAAAG AGATGCTTC |
| 105 | Reverse primer RP2 (39 nt) | | TTTTTTTTTT TTTTTTTTTT-S- |
| | | | CACGCCAAA GAGAT GCTTC |
| 130 | 5′ portion of RP2 | | TTTTTTTTTT TTTTTTTTTT |
| 131 | 3′ portion of RP2 | | CACGCCAAA GAGAT GCTTC |
| | Aptamers | | |
| | | Size | |
| | Name | (nt) | |
| 106 | MSA1 | 79 | TTACGTCAAG GTGTCACTCC |
| | | | CACTTTCCGG TTAATTTATG |
| | | | CTCTACCCGT CCACCTACCG |
| | | | GAAGCATCTC TTTGGCGTG |
| 107 | MSA3 | 79 | TTACGTCAAG GTGTCACTCC |
| | | | TACAGCGTCT GGTTGGTTTG |
| | | | GTTGGATCTT CGATCGCTGT |
| | | | GAAGCATCTC TTTGGCGTG |
| 108 | MSA5 | 79 | TTACGTCAAG GTGTCACTCC |
| | | | ACGGGTTTGG CGTCGGGCCT |
| | | | GGCGGGGGGA TAGTGCGGTG |
| | | | GAAGCATCTC TTTGGCGTG |
| 109 | MSA7 | 79 | TTACGTCAAG GTGTCACTCC |
| | | | TGGCTGTGGG GTTCGGGGTC |
| | | | ACTATTTGTC GGGAGGGGAG |
| | | | GAAGCATCTC TTTGGCGTG |
| 110 | MSA10 | 79 | TTACGTCAAG GTGTCACTCC |
| | | | GCGGGTTTGG CTCCGGGCCT |
| | | | GGCGTTGCGG TCTGCTCCCC |
| | | | GAAGCATCTC TTTGGCGTG |
| 111 | Mutant Control (MC) | 79 | TCTTTCGTGC CATCCTGCGG |
| | | | GTGGCTGTCC GAGGCTGGTG |
| | | | GCTCTGCAAG TGCCACGCTT |
| | | | TATCTGAGGT TCGCCGGTA |
| 112 | MSA52 | 79 | TTACGTCAAG GTGTCACTCC |
| | | | GTAGGGTTTG GCTCCGGGCC |
| | | | TGGCGTCGGT CGTCTCTCGC |
| | | | GAAGCATCTC TTTGGCGTG |
| 113 | MSA52-T1 | 68 | TTACGTCAAG GTGTCATTG |
| | | | GCTCCGGGCC TGGCGTCGGT |
| | | | CGTCTCTCGC GAAGCATCTC |
| | | | TTTGGCGTG |
| 114 | MSA52-T2 | 70 | TTACGTCAAG GTGTCACTCC |
| | | | GTAGGGTTTG CTGGCGTCGG |
| | | | TCGTCTCTCG CGAAGCATCT |
| | | | CTTTGGCGTG |
| 115 | MSA52-T3 | 58 | TTACGTCAAG GTGTCACTCC |
| | | | GTAGGGTTTG GCTCCGGGCC |
| | | | TGGCATCTCT TTGGCGTG |
| 116 | MSA52-T4 | 72 | TTACGTCAAG GTGTCACTCC |
| | | | GTAGGGTTTG GCTCCGGGCC |
| | | | TGGCGTCGGT CGCGAAGCAT |
| | | | CTCTTTGGCG TG |
| 117 | MSA52-T5 | 69 | ACGCCAAGGT GTCACTCCGT |
| | | | AGGGTTTGGC TCCGGGCCTG |
| | | | GCGTCGGTCG CGAAGCATCT |
| | | | CCTTGGCGT |
| 118 | CoV2-RBD-1 | 76 | ATCCAGAGTG ACGCAGCACC |
| | | | GACCTTGTGC TTTGGGAGTG |
| | | | CTGGTCCAAG GGCGTTAATG |
| | | | GACACGGTGG CTTAGT |
| 119 | Aptamer-1 | 76 | ATCCAGAGTG ACGCAGCATC |
| | | | GAGTGGCTTG TTTGTAATGT |
| | | | AGGGTTCCGG TCGTGGGTTG |
| | | | GACACGGTGG CTTAGT |
| 120 | CoV2-2 | 76 | ATCCAGAGTG ACGCAGCAGG |
| | | | GATGGGCTCC GGGCTACTGG |
| | | | CGAGGCTTCG GAACAACCGG |
| | | | ACACGGTGGC TTAGTA |
| 121 | S1P | 80 | GTCTTGACTA GTTACGCCTG |
| | | | GGAGGATTCG GCGCATGGGG |
| | | | ACGGGGGTGG CCCCCCCCCC |
| | | | TCTCATTCAG TTGGCGCCTC |
| 122 | nCOV-S1-A1 | 80 | AGCAGCACAG AGGTCAGATG |
| | | | CCGCAGGCAG CTGCCATTAG |
| | | | TCTCTATCCG TGACGGTATG |
| | | | CCTATGCGTG CTACCGTGAA |
| 123 | SARS2-AR10 | 45 | CCCGACCAGC CACCATCAGC |
| | | | AACTCTTCCG CGTCCATCCC TGCTG |
| 124 | B-MSA1 | 84 | Bio-TTTTTTTACG TCAAGGTGTC |
| | | | ACTCCCACTT TCCGGTTAAT |
| | | | TTATGCTCTA CCCGTCCACC |
| | | | TACCGGAAGC ATCTCTTTGG CGTG |
| 125 | B-MSA52 | 84 | Bio-TTTTTTTACG TCAAGGTGTC |
| | | | ACTCCGTAGG GTTTGGCTCC |
| | | | GGGCCTGGCG TCGGTCGTCT |
| | | | CTCGCGAAGC ATCTCTTTGG CGTG |
| 126 | B-S1P | 85 | Bio-TTTTTGTCTT GACTAGTTAC |
| | | | GCCTGGGAGG ATTCGGCGCA |
| | | | TGGGGACGGG GGTGGCCCCC |
| | | | CCCCCTCTCA TTCAGTTGGC GCCTC |
| 127 | B-nCOV-S1-A1 | 85 | Bio-TTTTTAGCAG CACAGAGGTC |
| | | | AGATGCCGCA GGCAGCTGCC |
| | | | ATTAGTCTCT ATCCGTGACG |
| | | | GTATGCCTAT GCGTGCTACC GTGAA |
| 128 | B-SARS2-AR10 | 50 | Bio-TTTTTCCCGA CCAGCCACCA |
| | | | TCAGCAACTC TTCCGCGTCC |
| | | | ATCCCTGCTG |
| 129 | B-A25 | 25 | Bio-AAAAAAAAAA AAAAAAAAAA |
| | | | AAAAA |

Sequences are written 5′-3′.
Abbreviations include: 40 bases randomregion (N40 in bold), S: non-amplifiable spacer.

TABLE 4
Affinity (K_{d}) summary of reported aptamers binding
to WHS and variant trimeric spike proteins

| Aptamer | K_{d} (nM) | | | | | |
|---|---|---|---|---|---|---|
| name | WHS | B.1.1.7S | B.1.351S | P.1S | B.1.617.2S | B.1.1.529S |
| MSA52 | 3.6 ± 0.4 | 3.8 ± 0.2 | 8.5 ± 0.8 | 10.2 ± 1.4 | 3.7 ± 0.4 | 6.2 ± 0.6 |
| | (2^{nd}) | (3^{rd}) | (2^{nd}) | (1^{st}) | (1^{st}) | (1^{st}) |
| MSA1 | 19.8 ± 2.6 | 1.2 ± 0.3 | >200 | 75.2 ± 6.1 | 6.8 ± 0.4 | 7.6 ± 0.5 |
| | | (1^{st}) | | | (3^{rd}) | (3^{rd}) |
| MSA3 | 5.5 ± 1.1 | 2.0 ± 0.3 | 36.4 ± 4.5 | 16.8 ± 1.5 | 12.8 ± 2.6 | 6.8 ± 0.8 |
| | (3^{rd}) | (2^{nd}) | | (2^{nd}) | | (2^{nd}) |
| MSA5 | 5.6 ± 0.6 | 4.2 ± 0.4 | 8.2 ± 0.8 | 52.3 ± 8.2 | 6.5 ± 1.5 | 15.4 ± 1.4 |
| | | | (1^{st}) | | (2^{nd}) | |
| MSA7 | 62.1 ± 8.2 | 8.4 ± 1.2 | 85.5 ± 9.8 | 33.5 ± 4.0 | 18.4 ± 0.8 | 22.4 ± 2.1 |
| | | | | (3^{rd}) | | |
| MSA10 | 69.4 ± 6.5 | 13.2 ± 3.0 | 75.6 ± 6.4 | 69.6 ± 5.7 | 22.1 ± 3.0 | 63.6 ± 5.2 |
| CoV2-RBD-1 | 37.4 ± 3.5 | 8.1 ± 0.4 | 78.9 ± 8.2 | 78.6 ± 7.4 | 28.2 ± 1.6 | 63.2 ± 4.6 |
| SP6 | 3.3 ± 0.4 | 11.5 ± 0.6 | 24.6 ± 2.2 | 72.4 ± 5.6 | 18.1 ± 2.8 | 41.2 ± 4.5 |
| | (1^{st}) | | (3^{rd}) | | | |
| S1P | 18.2 ± 2.5 | 5.6 ± 0.3 | 62.6 ± 7.8 | 65.8 ± 5.8 | 58.6 ± 4.8 | 34.3 ± 2.0 |
| nCoV-S1-A1 | 34.2 ± 5.0 | 28.4 ± 2.2 | 65.2 ± 5.8 | 43.5 ± 2.7 | 45.4 ± 3.9 | 72.6 ± 5.5 |
| SARS2-AR10 | 8.6 ± 1.3 | 6.4 ± 0.5 | 69.2 ± 7.9 | 42.4 ± 4.5 | 21.2 ± 2.5 | >200 |

### CITATIONS

1. Cutler, D. M. & Summers, L. H. The COVID-19 Pandemic and the $16 Trillion Virus. JAMA 324, 1495-1496 (2020).
2. Lazarevic, I., Pravica, V., Miljanovic, D. & Cupic, M. Immune Evasion of SARS-CoV-2 Emerging Variants: What Have We Learnt So Far? Viruses 13, 1192 (2021).
3. CDC COVID-19 Response Team. SARS-CoV-2 B.1.1.529 (Omicron) Variant—United States, Dec. 1-8, 2021. MMWR Morb. Mortal. Wkly. Rep. 70, 1731-1734 (2021).
4. Khan, P., Aufdembrink, L. M. & Engelhart, A. E. Isothermal SARS-CoV-2 Diagnostics: Tools for Enabling Distributed Pandemic Testing as a Means of Supporting Safe Reopenings. ACS Synth. Biol. 9, 2861-2880 (2020).
5. Dinnes, J. et al. Rapid, point-of-care antigen and molecular-based tests for diagnosis of SARS-CoV-2 infection. Cochrane Database Syst. Rev. 3, CD013705 (2021).
6. Parvu, V. et al. Clinical and experimental factors that affect the reported performance characteristics of rapid testing for SARS-CoV-2. medRxiv 2021.05.20.21257181 (2021) doi:10.1101/2021.05.20.21257181.
7. Stokes, W. et al. Real-World Clinical Performance of the Abbott Panbio with Nasopharyngeal, Throat and Saliva Swabs Among Symptomatic Individuals with COVID-19. medRxiv 2021.01.02.21249138 (2021) doi:10.1101/2021.01.02.21249138.
8. Tuerk, C. & Gold, L. Systematic evolution of ligands by exponential enrichment: RNA ligands to bacteriophage T4 DNA polymerase. Science 249, 505-510 (1990).
9. Ellington, A. D. & Szostak, J. W. In vitro selection of RNA molecules that bind specific ligands. Nature 346, 818-822 (1990).
10. McConnell, E. M., Cozma, I., Morrison, D. & Li, Y. Biosensors Made of Synthetic Functional Nucleic Acids Toward Better Human Health. Anal. Chem. 92, 327-344 (2020).
11. Kammer, M. N. et al. Quantification of Opioids in Urine Using an Aptamer-Based Free-Solution Assay. Anal. Chem. 91, 10582-10588 (2019).
12. Kim, S. H., Lee, J., Lee, B. H., Song, C.-S. & Gu, M. B. Specific detection of avian influenza H5N2 whole virus particles on lateral flow strips using a pair of sandwich-type aptamers. Biosens. Bioelectron. 134, 123-129 (2019).
13. Swensen, J. S. et al. Continuous, real-time monitoring of cocaine in undiluted blood serum via a microfluidic, electrochemical aptamer-based sensor. J. Am. Chem. Soc. 131, 4262-4266 (2009).
14. Liu, R., McConnell, E. M., Li, J. & Li, Y. Advances in functional nucleic acid based paper sensors. J. Mater. Chem. B8, 3213-3230 (2020).
15. Stanciu, L. A., Wei, Q., Barui, A. K. & Mohammad, N. Recent Advances in Aptamer-Based Biosensors for Global Health Applications. Annu. Rev. Biomed. Eng. (2021) doi:10.1146/annurev-bioeng-082020-035644.
16. Wang, T. et al. Development of nucleic acid aptamer-based lateral flow assays: A robust platform for cost-effective point-of-care diagnosis. Theranostics 11, 5174-5196 (2021).
17. Li, J. et al. Diverse high-affinity DNA aptamers for wild-type and B.1.1.7 SARS-CoV-2 spike proteins from a pre-structured DNA library. Nucleic Acids Res. 49, 7267-7279 (2021).
18. Song, Y. et al. Discovery of Aptamers Targeting the Receptor-Binding Domain of the SARS-CoV-2 Spike Glycoprotein. Anal. Chem. 92, 9895-9900 (2020).
19. Liu, X. et al. Neutralizing Aptamers Block S/RBD-ACE2 Interactions and Prevent Host Cell Infection. Angew. Chem. Int. Ed. 60, 10273-10278 (2021).
20. Schmitz, A. et al. A SARS-CoV-2 Spike Binding DNA Aptamer that Inhibits Pseudovirus Infection by an RBD-Independent Mechanism. Angew. Chem. Int. Ed. 60, 10279-10285 (2021).
21. Sun, M. et al. Aptamer Blocking Strategy Inhibits SARS-CoV-2 Virus Infection. Angew. Chem. Int. Ed. 60, 10266-10272 (2021).
22. Gupta, A. et al. A novel G-quadruplex aptamer-based spike trimeric antigen test for the detection of SARS-CoV-2. Mol. Ther.—Nucleic Acids 26, 321-332 (2021).
23. Yang, G. et al. Identification of SARS-CoV-2-against aptamer with high neutralization activity by blocking the RBD domain of spike protein 1. Signal Transduct. Target. Ther. 6, 1-4 (2021).
24. Peinetti, A. S. et al. Direct detection of human adenovirus or SARS-CoV-2 with ability to inform infectivity using DNA aptamer-nanopore sensors. Sci. Adv. 7, eabh2848 (2021).
25. Kacherovsky, N. et al. Discovery and Characterization of Spike N-Terminal Domain-Binding Aptamers for Rapid SARS-CoV-2 Detection. Angew. Chem. Int. Ed. 60, 21211-21215 (2021).
26. Ke, Z. et al. Structures and distributions of SARS-CoV-2 spike proteins on intact virions. Nature 588, 498-502 (2020).
27. Yao, H. et al. Molecular Architecture of the SARS-CoV-2 Virus. Cell 183, 730-738 (2020).
28. Schneider, C. A., Rasband, W. S. & Eliceiri, K. W. NIH Image to ImageJ: 25 years of image analysis. Nat. Methods 9, 671-675 (2012).
29. Martin, M. Cutadapt removes adapter sequences from high-throughput sequencing reads. EMBnet.journal 17, 10-12 (2011).
30. Edgar, R. C. Search and clustering orders of magnitude faster than BLAST. Bioinformatics 26, 2460-2461 (2010).
31. Edgar, R. C. MUSCLE: a multiple sequence alignment method with reduced time and space complexity. BMC Bioinformatics 5, 113 (2004).
32. Crooks, G. E., Hon, G., Chandonia, J.-M. & Brenner, S. E. WebLogo: A Sequence Logo Generator. Genome Res. 14, 1188-1190 (2004).
33. Stadlbauer, D. et al. SARS-CoV-2 Seroconversion in Humans: A Detailed Protocol for a Serological Assay, Antigen Production, and Test Setup. Curr. Protoc. Microbiol. 57, e100 (2020).
34. Amanat, F. et al. A serological assay to detect SARS-CoV-2 seroconversion in humans. Nat. Med. 26, 1033-1036 (2020).
35. Crawford, K. H. D. et al. Protocol and Reagents for Pseudotyping Lentiviral Particles with SARS-CoV-2 Spike Protein for Neutralization Assays. Viruses 12, 513 (2020).
Ellington, A. D. & Szostak, J. W., In vitro selection of RNA molecules that bind specific ligands. Nature 346, 818-822 (1990)
McConnell, E. M. et al., Biosensors Made of Synthetic Functional Nucleic Acids Toward Better Human Health. Anal. Chem. 92, 327-344 (2020)
U.S. Pat. No. 5,475,096
U.S. Pat. No. 5,270,163

1. A method of identifying or producing an aptamer capable of binding to at least two target analyte variants, the method comprising:
a) generating a mixture of systematic evolution of ligands by exponential enrichment (SELEX)-selected aptamers by at least one-round of SELEX with a SELEX-compatible aptamer pool against the at least two target analyte variants in parallel or sequentially;
b) sequencing the mixture of SELEX-selected aptamers that form complexes with each of the at least two target analyte variants by high-throughput sequencing;
c) aligning the mixture of SELEX-selected aptamers that form complexes with each of the at least two target analyte variants by sequence alignment analysis; and
d) identifying or producing the aptamer capable of binding to the at least two target analyte variants.

2. The method of claim 1, further comprising, prior to a), generating an aptamer pool compatible with SELEX.

3. The method of claim 1, wherein the SELEX-compatible aptamer pool comprises a plurality of nucleic acid molecules comprising at least one random nucleotide domain having an aptamer-like structure, the random nucleotide domain flanked by a 5′-end region and a 3-end region.

4. The method of claim 3, wherein the 5′-end region comprises a nucleotide sequence of SEQ ID NO: 102 or 103, and the 3′-end region comprises a nucleotide sequence of SEQ ID NO: 132 or the reverse complement of SEQ ID NO: 104 or 105.

5. The method of claim 1, wherein the SELEX-compatible aptamer pool comprises a plurality of nucleic acid molecules having the nucleotide sequence of SEQ ID NO: 101.

6. The method of claim 1, wherein the target analyte variant is a microorganism, a virus, and/or a molecule present in a microorganism or a virus.

7. The method of claim 1, wherein the target analyte variant is a protein, optionally the target analyte variant is a protein from SARS-CoV-2, optionally the target analyte variant is SARS-CoV-2 spike protein.

8. (canceled)

9. (canceled)

10. The method of claim 1, wherein a) comprises generating a mixture of SELEX-selected aptamers by at least three, four, five, six, seven, eight, or nine target analyte variants.

11. The method of claim 1, wherein the high-throughput sequencing comprises single-molecule real-time sequencing, ion semiconductor sequencing, pyrosequencing, sequencing by synthesis, combinatorial probe anchor synthesis sequencing, sequencing by ligation, nanopore sequencing, or GenapSys™ sequencing.

12. The method of claim 1, wherein the high-throughput sequencing comprises sequencing by synthesis.

13. An aptamer comprising a 5′-end region, a 3′-end region, and a nucleotide sequence selected from the group consisting of SEQ ID NOS: 2-100, a functional fragment, and functional variant thereof, or comprising a nucleotide sequence selected from the group consisting of SEQ ID NOS: 113-117, a functional fragment, and functional variant thereof.

14. The aptamer of claim 13 comprising a 5′-end region, a 3′-end region, and a nucleotide sequence selected from the group consisting of SEQ ID NOS: 2-10, a functional fragment, and functional variant thereof, optionally wherein the 5′-end region comprises a nucleotide sequence of SEQ ID NO: 102 or 103, and the 3′-end region comprises a nucleotide sequence of SEQ ID NO: 104 or 105, optionally the aptamer is for detecting SARS-CoV-2 spike protein.

15. The aptamer of claim 13 comprising a nucleotide sequence comprising SEQ ID NO: 116 or 117.

16. (canceled)

17. A method for detecting the presence of SARS-CoV-2 spike protein in a sample, the method comprising:
a) contacting the sample with an aptamer comprising a 5′-end region, a 3′-end region, and a nucleotide sequence selected from the group consisting of SEQ ID NOS: 1-100, a functional fragment, and functional variant thereof, or comprising a nucleotide sequence selected from the group consisting of SEQ ID NOS: 106-117, a functional fragment, and functional variant thereof, wherein the aptamer binds the spike protein; and
b) detecting the presence of the aptamer bound to the spike protein in the sample, wherein detection of bound aptamer indicates the presence of SARS-CoV-2 spike protein or variant thereof.

18. The method of claim 17, wherein the SARS-CoV-2 spike protein variant comprises B.1.1.7, B.1.351, P.1, B.1.429, B.1.617.1 B.1.617.2, B.1.617.2.1, or B.1.1.529 spike protein variant.

19. The method of claim 17, wherein the nucleotide sequence selected from the group consisting of SEQ ID NOS: 1-10, a functional fragment, and functional variant thereof.

20. The method of claim 17, wherein the nucleotide sequence comprises SEQ ID NO: 1, a functional fragment, or functional variant thereof.

21. The methods of claim 17, wherein the aptamer comprises a nucleotide sequence selected from the group consisting of SEQ ID NOS: 108, 112-117, a functional fragment, and functional variant thereof, or optionally the method detects SARS-CoV-2 infection in a subject, or optionally the sample is saliva, sputum, urine, blood, serum, other bodily fluids and/or secretions.

22. The method of claim 17, wherein the aptamer comprises a nucleotide sequence selected from the group consisting of SEQ ID NOS: 108, 112, 116, 117, a functional fragment, and functional variant thereof.

23. The method of claim 17, wherein the aptamer comprises a nucleotide sequence of SEQ ID NO: 112, a functional fragment, or functional variant thereof.

24. (canceled)

25. (canceled)

26. (canceled)


SEQUENCE LISTING (132 sequences)

SEQ ID NO: 1 - DNA, 40 bp, synthetic construct
  source 1..40: mol_type = other DNA
gtagggtttggctccgggcctggcgtcggtcgtctctcgc

SEQ ID NO: 2 - DNA, 39 bp, synthetic construct
  source 1..39: mol_type = other DNA
tagggtttggctccgggcctggcgtcggtcgtctctcgc

SEQ ID NO: 3 - DNA, 40 bp, synthetic construct
  source 1..40: mol_type = other DNA
atagggtttggctccgggcctggcgtcggtcgtctctcgc

SEQ ID NO: 4 - DNA, 40 bp, synthetic construct
  source 1..40: mol_type = other DNA
gtagggtttggctccgggcctggcgtcggtcgtctctcgt

SEQ ID NO: 5 - DNA, 40 bp, synthetic construct
  source 1..40: mol_type = other DNA
ttagggtttggctccgggcctggcgtcggtcgtctctcgc

SEQ ID NO: 6 - DNA, 40 bp, synthetic construct
  source 1..40: mol_type = other DNA
gtagggtttggctccgggcctggcgtcggtcgtctttcgc

SEQ ID NO: 7 - DNA, 40 bp, synthetic construct
  source 1..40: mol_type = other DNA
gtagggtttggctccgggcctggcgtcggtcgtctcttgc

SEQ ID NO: 8 - DNA, 39 bp, synthetic construct
  source 1..39: mol_type = other DNA
gcgggtttggctccgggcctggcgtcggtcgtctctcgc

SEQ ID NO: 9 - DNA, 40 bp, synthetic construct
  source 1..40: mol_type = other DNA
gtagggtttggctccgggcctggcgtcggtcgtctcccgc

SEQ ID NO: 10 - DNA, 40 bp, synthetic construct
  source 1..40: mol_type = other DNA
gcagggtttggctccgggcctggcgtcggtcgtctctcgc

SEQ ID NO: 11 - DNA, 40 bp, synthetic construct
  source 1..40: mol_type = other DNA
ctagggtttggctccgggcctggcgtcggtcgtctctcgc

SEQ ID NO: 12 - DNA, 40 bp, synthetic construct
  source 1..40: mol_type = other DNA
gtagggtttggctccgggcctggcgtcggttgtctctcgc

SEQ ID NO: 13 - DNA, 40 bp, synthetic construct
  source 1..40: mol_type = other DNA
gtagggtttggctccgggcctggcgtcggtcatctctcgc

SEQ ID NO: 14 - DNA, 40 bp, synthetic construct
  source 1..40: mol_type = other DNA
gtagggtttggctccgggcctggcgtcggtcgtccctcgc

SEQ ID NO: 15 - DNA, 40 bp, synthetic construct
  source 1..40: mol_type = other DNA
gtggggtttggctccgggcctggcgtcggtcgtctctcgc

SEQ ID NO: 16 - DNA, 40 bp, synthetic construct
  source 1..40: mol_type = other DNA
gtagggtttggctccgggcctggcgtaggtcgtctctcgc

SEQ ID NO: 17 - DNA, 40 bp, synthetic construct
  source 1..40: mol_type = other DNA
gaagggtttggctccgggcctggcgtcggtcgtctctcgc

SEQ ID NO: 18 - DNA, 40 bp, synthetic construct
  source 1..40: mol_type = other DNA
gtagggtttggctccgggcctggcgtcggtcgcctctcgc

SEQ ID NO: 19 - DNA, 40 bp, synthetic construct
  source 1..40: mol_type = other DNA
gtagggtttggctccgggcctggcgtcggtcgtctctcga

SEQ ID NO: 20 - DNA, 39 bp, synthetic construct
  source 1..39: mol_type = other DNA
acgggtttggctccgggcctggcgtcggtcgtctctcgc

SEQ ID NO: 21 - DNA, 40 bp, synthetic construct
  source 1..40: mol_type = other DNA
gtagggtttggctccgggcctggcgtcggtcgtctctctc

SEQ ID NO: 22 - DNA, 40 bp, synthetic construct
  source 1..40: mol_type = other DNA
gtagggtttggctccgggcctggcgtcggtcgtttctcgc

SEQ ID NO: 23 - DNA, 40 bp, synthetic construct
  source 1..40: mol_type = other DNA
atagggtttggctccgggcctggcgtcggtcgtctctcgt

SEQ ID NO: 24 - DNA, 38 bp, synthetic construct
  source 1..38: mol_type = other DNA
tgggtttggctccgggcctggcgtcggtcgtctctcgc

SEQ ID NO: 25 - DNA, 40 bp, synthetic construct
  source 1..40: mol_type = other DNA
gtagggtttggctcagggcctggcgtcggtcgtctctcgc

SEQ ID NO: 26 - DNA, 39 bp, synthetic construct
  source 1..39: mol_type = other DNA
gtagggtttggctccgggcctggcgtcggtcgtctcctc

SEQ ID NO: 27 - DNA, 40 bp, synthetic construct
  source 1..40: mol_type = other DNA
gtagggtttggctccgggcctggcgtcggtcctctctcgc

SEQ ID NO: 28 - DNA, 40 bp, synthetic construct
  source 1..40: mol_type = other DNA
gtagggtttggctccgggcctggcgacggtcgtctctcgc

SEQ ID NO: 29 - DNA, 40 bp, synthetic construct
  source 1..40: mol_type = other DNA
gtagggtttggctccgggcctggcgtcggtcttctctcgc

SEQ ID NO: 30 - DNA, 39 bp, synthetic construct
  source 1..39: mol_type = other DNA
cagggtttggctccgggcctggcgtcggtcgtctctcgc

SEQ ID NO: 31 - DNA, 38 bp, synthetic construct
  source 1..38: mol_type = other DNA
agggtttggctccgggcctggcgtcggtcgtctctcgc

SEQ ID NO: 32 - DNA, 40 bp, synthetic construct
  source 1..40: mol_type = other DNA
gtagggtttggctccggacctggcgtcggtcgtctctcgc

SEQ ID NO: 33 - DNA, 39 bp, synthetic construct
  source 1..39: mol_type = other DNA
gtagggtttggctccgggcctgcgtcggtcgtctctcgc

SEQ ID NO: 34 - DNA, 40 bp, synthetic construct
  source 1..40: mol_type = other DNA
gtagggtttggctccgggcctggcgtcggtcgtctctcac

SEQ ID NO: 35 - DNA, 40 bp, synthetic construct
  source 1..40: mol_type = other DNA
gtagggtttggctccgggcctggcgtcggtcgtctctcgg

SEQ ID NO: 36 - DNA, 40 bp, synthetic construct
  source 1..40: mol_type = other DNA
actgggtttggctccgggcctggcgtcggtcgtctctcgc

SEQ ID NO: 37 - DNA, 40 bp, synthetic construct
  source 1..40: mol_type = other DNA
gtagggtttggctccgggcctggcgtcggtcgtctctagc

SEQ ID NO: 38 - DNA, 40 bp, synthetic construct
  source 1..40: mol_type = other DNA
gtagggcttggctccgggcctggcgtcggtcgtctctcgc

SEQ ID NO: 39 - DNA, 40 bp, synthetic construct
  source 1..40: mol_type = other DNA
gtagggtttggctccgggcctggcgtcggtcgtctctccc

SEQ ID NO: 40 - DNA, 40 bp, synthetic construct
  source 1..40: mol_type = other DNA
atggggtttggctccgggcctggcgtcggtcgtctctcgc

SEQ ID NO: 41 - DNA, 40 bp, synthetic construct
  source 1..40: mol_type = other DNA
gtagggtttggctccgggcctggcgtcgttcgtctctcgc

SEQ ID NO: 42 - DNA, 40 bp, synthetic construct
  source 1..40: mol_type = other DNA
gtagggtttggctccgggcctagcgtcggtcgtctctcgc

SEQ ID NO: 43 - DNA, 40 bp, synthetic construct
  source 1..40: mol_type = other DNA
gtagggtttggctccgggcctggcgtcggtagtctctcgc

SEQ ID NO: 44 - DNA, 40 bp, synthetic construct
  source 1..40: mol_type = other DNA
ggagggtttggcatcgggcctggcgtcggtcgtctctcgc

SEQ ID NO: 45 - DNA, 40 bp, synthetic construct
  source 1..40: mol_type = other DNA
gtagggtttggctccgggcctggcttcggtcgtctctcgc

SEQ ID NO: 46 - DNA, 40 bp, synthetic construct
  source 1..40: mol_type = other DNA
gtagggtttggctccgggcctggcgccggtcgtctctcgc

SEQ ID NO: 47 - DNA, 40 bp, synthetic construct
  source 1..40: mol_type = other DNA
gtagggtttggctccgggcccggcgtcggtcgtctctcgc

SEQ ID NO: 48 - DNA, 39 bp, synthetic construct
  source 1..39: mol_type = other DNA
tggggtttggctccgggcctggcgtcggtcgtctctcgc

SEQ ID NO: 49 - DNA, 40 bp, synthetic construct
  source 1..40: mol_type = other DNA
acagggtttggctccgggcctggcgtcggtcgtctctcgc

SEQ ID NO: 50 - DNA, 40 bp, synthetic construct
  source 1..40: mol_type = other DNA
gtagggtctggctccgggcctggcgtcggtcgtctctcgc

SEQ ID NO: 51 - DNA, 40 bp, synthetic construct
  source 1..40: mol_type = other DNA
gtagggtttggctccgggcctggcgtcgatcgtctctcgc

SEQ ID NO: 52 - DNA, 40 bp, synthetic construct
  source 1..40: mol_type = other DNA
gtagggtttggctccgggcctggcgtcggtcgtctcccgt

SEQ ID NO: 53 - DNA, 40 bp, synthetic construct
  source 1..40: mol_type = other DNA
atagggtttggctccgggcctggcgtcggtcgtttctcgc

SEQ ID NO: 54 - DNA, 40 bp, synthetic construct
  source 1..40: mol_type = other DNA
atagggtttggctccgggcctggcgtcggtcttctctcgc

SEQ ID NO: 55 - DNA, 40 bp, synthetic construct
  source 1..40: mol_type = other DNA
gtagggtttggctacgggcctggcgtcggtcgtctctcgc

SEQ ID NO: 56 - DNA, 40 bp, synthetic construct
  source 1..40: mol_type = other DNA
gtagggtttggctccgggcctggcgtcggtcgtgtctcgc

SEQ ID NO: 57 - DNA, 39 bp, synthetic construct
  source 1..39: mol_type = other DNA
tagggtttggctccgggcctggcgtcgttcgtctctcgc

SEQ ID NO: 58 - DNA, 39 bp, synthetic construct
  source 1..39: mol_type = other DNA
tagggtttggctccgggcctggcgtcggtcgtctctcgt

SEQ ID NO: 59 - DNA, 43 bp, synthetic construct
  source 1..43: mol_type = other DNA
tcgggggggtttggctccgggcctggcgtcggtcgtctctcgc

SEQ ID NO: 60 - DNA, 39 bp, synthetic construct
  source 1..39: mol_type = other DNA
tagggtttggctccgggcctggcgtcggtcgtctttcgc

SEQ ID NO: 61 - DNA, 40 bp, synthetic construct
  source 1..40: mol_type = other DNA
gtagggtttggctccgggcctggcgtcggccgtctctcgc

SEQ ID NO: 62 - DNA, 40 bp, synthetic construct
  source 1..40: mol_type = other DNA
gttgggtttggctccgggcctggcgtcggtcgtctctcgc

SEQ ID NO: 63 - DNA, 43 bp, synthetic construct
  source 1..43: mol_type = other DNA
tccgtagggtttggctccgggcctggcgtcggtcgtctctcgc

SEQ ID NO: 64 - DNA, 40 bp, synthetic construct
  source 1..40: mol_type = other DNA
gtagggtttggccccgggcctggcgtcggtcgtctctcgc

SEQ ID NO: 65 - DNA, 40 bp, synthetic construct
  source 1..40: mol_type = other DNA
gtagggtttggctccaggcctggcgtcggtcgtctctcgc

SEQ ID NO: 66 - DNA, 40 bp, synthetic construct
  source 1..40: mol_type = other DNA
gtagggttcggctccgggcctggcgtcggtcgtctctcgc

SEQ ID NO: 67 - DNA, 38 bp, synthetic construct
  source 1..38: mol_type = other DNA
gtagggtttggctccgggcctggcgtcggtcgtctctc

SEQ ID NO: 68 - DNA, 39 bp, synthetic construct
  source 1..39: mol_type = other DNA
tcgggtttggctccgggcctggcgtcggtcgtctctcgc

SEQ ID NO: 69 - DNA, 40 bp, synthetic construct
  source 1..40: mol_type = other DNA
gtagggtttggctccgggcctggcgtcggacgtctctcgc

SEQ ID NO: 70 - DNA, 40 bp, synthetic construct
  source 1..40: mol_type = other DNA
gtagggtttggctccgggcctggcgttggtcgtctctcgc

SEQ ID NO: 71 - DNA, 40 bp, synthetic construct
  source 1..40: mol_type = other DNA
gtagggtttggctccgggcctggcgtcggtcgtctatcgc

SEQ ID NO: 72 - DNA, 40 bp, synthetic construct
  source 1..40: mol_type = other DNA
atagggtttggctccgggcctggcgtcggttgtctctcgc

SEQ ID NO: 73 - DNA, 39 bp, synthetic construct
  source 1..39: mol_type = other DNA
gcgggtttggctccgggcctggcgtcggtcgtctctcgt

SEQ ID NO: 74 - DNA, 40 bp, synthetic construct
  source 1..40: mol_type = other DNA
gtagggtttggctccgggcctggcgtcagtcgtctctcgc

SEQ ID NO: 75 - DNA, 40 bp, synthetic construct
  source 1..40: mol_type = other DNA
gtagggtttggcttcgggcctggcgtcggtcgtctctcgc

SEQ ID NO: 76 - DNA, 40 bp, synthetic construct
  source 1..40: mol_type = other DNA
gtagggtttggctccgggcctggcgtcggtcgtcgctcgc

SEQ ID NO: 77 - DNA, 40 bp, synthetic construct
  source 1..40: mol_type = other DNA
gtagggtttggctccgggcctggcgtcggtcgactctcgc

SEQ ID NO: 78 - DNA, 40 bp, synthetic construct
  source 1..40: mol_type = other DNA
gtagggtttggctccgggcctggcgtcggtcgtcactcgc

SEQ ID NO: 79 - DNA, 40 bp, synthetic construct
  source 1..40: mol_type = other DNA
gtagggtttggctccgggcctggcgtctgtcgtctctcgc

SEQ ID NO: 80 - DNA, 40 bp, synthetic construct
  source 1..40: mol_type = other DNA
atcgggtttggctccgggcctggcgtcggtcgtctctcgc

SEQ ID NO: 81 - DNA, 39 bp, synthetic construct
  source 1..39: mol_type = other DNA
gtagggtttggctccgggcctggcgtcggtcgtctcccc

SEQ ID NO: 82 - DNA, 39 bp, synthetic construct
  source 1..39: mol_type = other DNA
tagggtttggctccgggcctggcgtcggtcgtctcttgc

SEQ ID NO: 83 - DNA, 40 bp, synthetic construct
  source 1..40: mol_type = other DNA
ttagggtttggctccgggcctggcgtcggtcgtctctcgt

SEQ ID NO: 84 - DNA, 40 bp, synthetic construct
  source 1..40: mol_type = other DNA
gtagggtttggctctgggcctggcgtcggtcgtctctcgc

SEQ ID NO: 85 - DNA, 38 bp, synthetic construct
  source 1..38: mol_type = other DNA
ggggtttggctccgggcctggcgtcggtcgtctctcgc

SEQ ID NO: 86 - DNA, 39 bp, synthetic construct
  source 1..39: mol_type = other DNA
atgggtttggctccgggcctggcgtcggtcgtctctcgc

SEQ ID NO: 87 - DNA, 40 bp, synthetic construct
  source 1..40: mol_type = other DNA
gtagggtttggctccgggcctgacgtcggtcgtctctcgc

SEQ ID NO: 88 - DNA, 38 bp, synthetic construct
  source 1..38: mol_type = other DNA
tgggattggctccgggcctggcgtcggtcgtctctcgc

SEQ ID NO: 89 - DNA, 40 bp, synthetic construct
  source 1..40: mol_type = other DNA
gtagggtttggctccgggtctggcgtcggtcgtctctcgc

SEQ ID NO: 90 - DNA, 40 bp, synthetic construct
  source 1..40: mol_type = other DNA
atagggtttggctccgggcctggcgtcggtcatctctcgc

SEQ ID NO: 91 - DNA, 41 bp, synthetic construct
  source 1..41: mol_type = other DNA
cactgggtttggctccgggcctggcgtcggtcgtctctcgc

SEQ ID NO: 92 - DNA, 40 bp, synthetic construct
  source 1..40: mol_type = other DNA
gcagggtttggctccgggcctggcgtcggtcgtctctcgt

SEQ ID NO: 93 - DNA, 38 bp, synthetic construct
  source 1..38: mol_type = other DNA
ggggattggctccgggcctggcgtcggtcgtctctcgc

SEQ ID NO: 94 - DNA, 40 bp, synthetic construct
  source 1..40: mol_type = other DNA
gtagggtttggctccgggactggcgtcggtcgtctctcgc

SEQ ID NO: 95 - DNA, 40 bp, synthetic construct
  source 1..40: mol_type = other DNA
gtagggtttggctccgggcctggcgtcggtcgtctccccc

SEQ ID NO: 96 - DNA, 39 bp, synthetic construct
  source 1..39: mol_type = other DNA
tagggtttggctccgggcctggcgtcggccgtctctcgc

SEQ ID NO: 97 - DNA, 39 bp, synthetic construct
  source 1..39: mol_type = other DNA
tagggtttggctccgggcctggcgtcggtcatctctcgc

SEQ ID NO: 98 - DNA, 40 bp, synthetic construct
  source 1..40: mol_type = other DNA
gtagggtttggctccgggcctggtgtcggtcgtctctcgc

SEQ ID NO: 99 - DNA, 40 bp, synthetic construct
  source 1..40: mol_type = other DNA
gtagggtttggctcctggcctggcgtcggtcgtctctcgc

SEQ ID NO: 100 - DNA, 40 bp, synthetic construct
  source 1..40: mol_type = other DNA
gtagggattggctccgggcctggcgtcggtcgtctctcgc

SEQ ID NO: 101 - DNA, 79 bp, synthetic construct
  source 1..79: mol_type = other DNA
  misc_feature 21..60: note = n is a, c, g, or t
ttacgtcaaggtgtcactccnnnnnnnnnnnnnnnnnnnnnnnnnnnnnnnnnnnnnnnngaagcatctctttggcgtg

SEQ ID NO: 102 - DNA, 20 bp, synthetic construct
  source 1..20: mol_type = other DNA
ttacgtcaaggtgtcactcc

SEQ ID NO: 103 - DNA, 20 bp, synthetic construct
  source 1..20: mol_type = other DNA
  modified_base 1: mod_base = OTHER; note = fluorescein thymidine
ttacgtcaaggtgtcactcc

SEQ ID NO: 104 - DNA, 19 bp, synthetic construct
  source 1..19: mol_type = other DNA
cacgccaaagagatgcttc

SEQ ID NO: 105 - DNA, 39 bp, synthetic construct
  source 1..39: mol_type = other DNA
  misc_feature 20^21: note = non amplifiable spacer
ttttttttttttttttttttcacgccaaagagatgcttc

SEQ ID NO: 106 - DNA, 79 bp, synthetic construct
  source 1..79: mol_type = other DNA
ttacgtcaaggtgtcactcccactttccggttaatttatgctctacccgtccacctaccggaagcatctctttggcgtg

SEQ ID NO: 107 - DNA, 79 bp, synthetic construct
  source 1..79: mol_type = other DNA
ttacgtcaaggtgtcactcctacagcgtctggttggtttggttggatcttcgatcgctgtgaagcatctctttggcgtg

SEQ ID NO: 108 - DNA, 79 bp, synthetic construct
  source 1..79: mol_type = other DNA
ttacgtcaaggtgtcactccacgggtttggcgtcgggcctggcggggggatagtgcggtggaagcatctctttggcgtg

SEQ ID NO: 109 - DNA, 79 bp, synthetic construct
  source 1..79: mol_type = other DNA
ttacgtcaaggtgtcactcctggctgtggggttcggggtcactatttgtcgggaggggaggaagcatctctttggcgtg

SEQ ID NO: 110 - DNA, 79 bp, synthetic construct
  source 1..79: mol_type = other DNA
ttacgtcaaggtgtcactccgcgggtttggctccgggcctggcgttgcggtctgctccccgaagcatctctttggcgtg

SEQ ID NO: 111 - DNA, 79 bp, synthetic construct
  source 1..79: mol_type = other DNA
tctttcgtgccatcctgcgggtggctgtccgaggctggtggctctgcaagtgccacgctttatctgaggttcgccggta

SEQ ID NO: 112 - DNA, 79 bp, synthetic construct
  source 1..79: mol_type = other DNA
ttacgtcaaggtgtcactccgtagggtttggctccgggcctggcgtcggtcgtctctcgcgaagcatctctttggcgtg

SEQ ID NO: 113 - DNA, 68 bp, synthetic construct
  source 1..68: mol_type = other DNA
ttacgtcaaggtgtcattggctccgggcctggcgtcggtcgtctctcgcgaagcatctctttggcgtg

SEQ ID NO: 114 - DNA, 70 bp, synthetic construct
  source 1..70: mol_type = other DNA
ttacgtcaaggtgtcactccgtagggtttgctggcgtcggtcgtctctcgcgaagcatctctttggcgtg

SEQ ID NO: 115 - DNA, 58 bp, synthetic construct
  source 1..58: mol_type = other DNA
ttacgtcaaggtgtcactccgtagggtttggctccgggcctggcatctctttggcgtg

SEQ ID NO: 116 - DNA, 72 bp, synthetic construct
  source 1..72: mol_type = other DNA
ttacgtcaaggtgtcactccgtagggtttggctccgggcctggcgtcggtcgcgaagcatctctttggcgtg

SEQ ID NO: 117 - DNA, 69 bp, synthetic construct
  source 1..69: mol_type = other DNA
acgccaaggtgtcactccgtagggtttggctccgggcctggcgtcggtcgcgaagcatctccttggcgt

SEQ ID NO: 118 - DNA, 76 bp, synthetic construct
  source 1..76: mol_type = other DNA
atccagagtgacgcagcaccgaccttgtgctttgggagtgctggtccaagggcgttaatggacacggtggcttagt

SEQ ID NO: 119 - DNA, 76 bp, synthetic construct
  source 1..76: mol_type = other DNA
atccagagtgacgcagcatcgagtggcttgtttgtaatgtagggttccggtcgtgggttggacacggtggcttagt

SEQ ID NO: 120 - DNA, 76 bp, synthetic construct
  source 1..76: mol_type = other DNA
atccagagtgacgcagcagggatgggctccgggctactggcgaggcttcggaacaaccggacacggtggcttagta

SEQ ID NO: 121 - DNA, 80 bp, synthetic construct
  source 1..80: mol_type = other DNA
gtcttgactagttacgcctgggaggattcggcgcatggggacgggggtggcccccccccctctcattcagttggcgcctc

SEQ ID NO: 122 - DNA, 80 bp, synthetic construct
  source 1..80: mol_type = other DNA
agcagcacagaggtcagatgccgcaggcagctgccattagtctctatccgtgacggtatgcctatgcgtgctaccgtgaa

SEQ ID NO: 123 - DNA, 45 bp, synthetic construct
  source 1..45: mol_type = other DNA
cccgaccagccaccatcagcaactcttccgcgtccatccctgctg

SEQ ID NO: 124 - DNA, 84 bp, synthetic construct
  source 1..84: mol_type = other DNA
  modified_base 1: mod_base = OTHER; note = biotinylated thymidine
tttttttacgtcaaggtgtcactcccactttccggttaatttatgctctacccgtccacctaccggaagcatctctttggcgtg

SEQ ID NO: 125 - DNA, 84 bp, synthetic construct
  source 1..84: mol_type = other DNA
  modified_base 1: mod_base = OTHER; note = biotinylated thymidine
tttttttacgtcaaggtgtcactccgtagggtttggctccgggcctggcgtcggtcgtctctcgcgaagcatctctttggcgtg

SEQ ID NO: 126 - DNA, 85 bp, synthetic construct
  source 1..85: mol_type = other DNA
  modified_base 1: mod_base = OTHER; note = biotinylated thymidine
tttttgtcttgactagttacgcctgggaggattcggcgcatggggacgggggtggcccccccccctctcattcagttggcgcctc

SEQ ID NO: 127 - DNA, 85 bp, synthetic construct
  source 1..85: mol_type = other DNA
  modified_base 1: mod_base = OTHER; note = biotinylated thymidine
tttttagcagcacagaggtcagatgccgcaggcagctgccattagtctctatccgtgacggtatgcctatgcgtgctaccgtgaa

SEQ ID NO: 128 - DNA, 50 bp, synthetic construct
  source 1..50: mol_type = other DNA
  modified_base 1: mod_base = OTHER; note = biotinylated thymidine
tttttcccgaccagccaccatcagcaactcttccgcgtccatccctgctg

SEQ ID NO: 129 - DNA, 25 bp, synthetic construct
  source 1..25: mol_type = other DNA
  modified_base 1: mod_base = OTHER; note = biotinylated deoxyadenosine
aaaaaaaaaaaaaaaaaaaaaaaaa

SEQ ID NO: 130 - DNA, 20 bp, synthetic construct
  source 1..20: mol_type = other DNA
tttttttttttttttttttt

SEQ ID NO: 131 - DNA, 19 bp, synthetic construct
  source 1..19: mol_type = other DNA
cacgccaaagagatgcttc

SEQ ID NO: 132 - DNA, 19 bp, synthetic construct
  source 1..19: mol_type = other DNA
gaagcatctctttggcgtg